SEQUENCE LISTING (18 sequences)

SEQ ID NO: 1 - protein, 592 aa, Candida albicans
MNQTLVRMMNMPNEDPNVSSEEAAAANVATIHNIKNEDSMHYQFGHHQPSIYDQQQQQQQQHHHQLPHHPIHYHGIAQQQQSQLPHFAMHPSAGVNNNQHLNHQPYQQMSHYNAQQMHQQQLHHQLMTPNPYQQHFQQQMHPQLHHEDHLNMHFNPMSYPQQQQQQQQQQQQQQQHLHHFGHQIPAPPAQQGPTPQQPHLHQQIPHPLSHHQTPQPTPQPLAQQQSPQPARQPRQTKKQKQQAQNQDQADAQSQAQQHHMAMMARANQNDMLESSTRKVAPRSSDLFRVGPPFSISKQHQPVYCVGTDMPVTPLLHARIDRGFEMGETGSWIGYKRNYFTLVASFTLQDFDFEKFIGNKFYTYDKVNNKVNGFPPHHPSHPQNQPQNHPGHPHHNQHAGESRVPISYFAIRLVAKCSDEDVAISLIQHTAKRDKGPQFPPPIYPAVPSELPDHETVKVSCNKRNNNKIETMNKIFYFDRGNYYQEYNLDSYKDQSILKSYPSQSISKVARFERIQFTSSIRVKSTNTTARYFTLHVELLGIIEDEDLQIQPILLSSIESPPLIVRGRSPSSYHKDRTSGYRATNTPTPTPPQ

SEQ ID NO: 2 - protein, 627 aa, Saccharomyces cerevisiae
MNEMENTDPVLQDDLVSKYERELSTEQEEDTPVILTQLNEDGTTSNYFDKRKLKIAPRSTLQFKVGPPFELVRDYCPVVESHTGRTLDLRIIPRIDRGFDHIDEEWVGYKRNYFTLVSTFETANCDLDTFLKSSFDLLVEDSSVESRLRVQYFAIKIKAKNDDDDTEINLVQHTAKRDKGPQFCPSVCPLVPSPLPKHQIIREASNVRNITKMKKYDSTFYLHRDHVNYEEYGVDSLLFSYPEDSIQKVARYERVQFASSISVKKPSQQNKHFSLHVILGAVVDPDTFHGENPGIPYDELALKNGSKGMFVYLQEMKTPPLIIRGRSPSNYASSQRITVRTPSSVNSSQNSTKRKMPSMAQPLNESCLNARPSKRRSKVALGAPNSGASISPIKSRQSTPMEASKENEDPFFRPNKRVETLEHIQNKLGALKNQCPDSSLKYPSSSSRGMEGCLEKEDLVYSSSFSVNMKQIELKPARSFEHENIFKVGSLALKKINELPHENYDITIEKKSMEQNYLRPEIGSRSECKTSYGNELSLSNISFSILPNSAENFHLETALFPATEEDVNRTFSRILETGSFQNYYQKMDAENADRVYSKGVKLIASGTLPSGIFNREELFEEDSFYKY

SEQ ID NO: 3 - DNA, 2838 bp, Candida albicans
ttttgtggcgattttcactttctcttcaaaattcaaattgtccaaaaacttttctctgttgctcatctttttttttatataaactcaacctttatcttattttaaagattaagtttgtgtttaataggtttttctcccacaacagtttatcaacgctttccccgtttttatatcacttcattgagtccccgttcactagtaccagcttgtcaacttttaaaaaaaaagaaattcttacagtagtgcacacttgctcaaaaaaaccgaattttcaatcccatcacatcaaacggaacgactgaggaaaaaaaaaagacaacattaaaattttttttcttttttttttttttttcctttcccatctccatattaccatcttcctttcaagctttatacttcaggagatatcatatcgttactatcaaggtatttagcaaaccaacacatatacctttgcgtgataccagattcttgtcgacaaccgaaaaaaaaagaaagataatttcacaaagtccagacagcatcaacaacaaaaagttttttttgtttccctttttttttacgacaccaaccgatatatatgaatcaaactcttgtcagaatgatgaacatgcccaacgaagatcctaacgtatcgagtgaagaagctgccgctgctaatgttgcaactattcacaatatcaaaaacgaagactccatgcactatcaatttggacaccatcagccttcaatttatgatcaacaacaacaacaacaacagcaacaccaccaccagcttcctcaccatcctattcactatcatggtattgctcagcaacaacagtcccagcttcctcatttcgctatgcatccatcagctggtgtcaacaacaaccaacatttgaaccaccagccataccagcagatgtcgcactacaatgcccaacaaatgcatcaacaacaattacaccatcagttgatgaccccaaacccataccaacaacatttccaacaacaaatgcaccctcaattacaccacgaggaccatttgaatatgcatttcaacccaatgtcctatccacaacaacaacagcagcaacagcagcagcaacaacaacaacaacaacatttgcaccatttcggtcaccaaattccagccccccctgctcaacaaggcccaacacctcagcagccacatttgcatcaacaaattcctcatccattatcacaccaccaaactccccaaccaacaccacaacctttggctcagcaacaactgcctcaaccagcaagacaaccacgtcaaaccaagaaacaaaaacaacaagcacaaaaccaagaccaagctgacgctcaatcacaagcccaacagcaccatatggctatgatggctcgtgctaaccaaaacgatatgttggaatcgtcgaccagaaaagtcgcacctagatctagcgatttgttccgtgttgggcctccattttccatcagcaaacaacaccagccagtatattgtgtggggaccgatatgccagtgacaccattgttgcatgcccgtattgatagagggtttgagatgggcgaaaccggttcttggattggctataagcgtaactactttaccttggttgcatcgtttacattgcaagacttcgattttgaaaagttcatcggcaacaagttctacacttacgacaaggtcaacaacaaggttaatggtttcccacctcatcaccctagccacccacaaaaccagccccaaaaccatcccggccatccacaccacaaccaacacgctggcgaactgagagttccaattagttattttgcaattaggttagtggccaagtgttctgatgaagatgttgccatctccttgatccagcacactgctaaaagagataaaggtccacagttcccaccaccaatctaccctgcagttccttcagagttgcctgaccacgagactgtcaaagtaagttgtaacaaacgtaacaacaacaagatcgaaactatgaataaaatcttttactttgataggggaaactactaccaagagtacaatttggatagctacaaggaccagtcgatattgaaaagctaccctagtcaatccatttccaaggtggctagatttgaaagaatccaattcaccagctctattagagttaaaagcaccaacacaactgctagatactttaccttgcatgtcgaattattgggtataatcgaggacgaggacttgcagatccaaccaatcttattaagttcaattgaatcaccgcctttaatcgtcagaggcagatctccttcaagctaccacaaagatagaacttctgggtatagggctacaaacaccccaacccctactcctccacagtaagcaaaactatagatttttttttttttttgatgatgacgaagaaaggttgattttaaagtttaaagtaaataaaaaaattttgtattataattacacaaaagatcacaaaatatatacactgatacatatataaagcttttatttgtaatagtaatcttatttatagccgtaactgccccatccccacaaaatatcagagccaggtgtcgtgtaccaatcacccgcaccaaaaaaatttttaggtttctccccactacaggagccaacgtaagaaaaaaaaaatatttgattctcatcaaataataattgtgacattaataagacattgctgaagtatgacccaatctaatagtgactcgtcaagtactggcgacgtacccaatatcaacactttgctaataccgtcgatctcaaaaccagtatactcacgtatccctatcccgggccatttaagcaatgatcaatccacccccattgag

SEQ ID NO: 4 - DNA, 3400 bp, Candida albicans
tgcctgagttcaagatggtctataaaagcatgtattgggtatgtcaatctgaacaatacaaactaaacaagacttggaaatttcatattaatttgctttcattgtttgtgttttttcttccccaattgttgtttagtgaaatatccgataagtttaaaatttcagaatagtgattttttttttcaccaaccacaaccataattaagctttcttttgttaccaaaagtatcaaacctaaaacaaaactagctaataaggtttagattgttaaagaattgtgtttttttttttttttttggcttggaattttgctattctacaagaaataaccatcttcaagcgccaatttaccaccattttaagtttaaactacattttttttgttgttgggttgttatctcatatcaattcaaaacttaatcttcaccctggaaattacaagagtacttaactgggtctttttcctcgacatttgcctccccaaacctttttcatcctcatccgctaatactttcgttggtaaatctggatttaaaaactacttatctccagtggaaaaaaaaaataaaagctgcaaaaacagacaaaagtcaaattattttaaccctcaccctgaatattctcaaaataacctgggaataagaattcttattagtataaccaatcttgggcaagagattttccaaggacatttttgagatcttgttaaaacgacatttttgacatttatttttttttttttgtttattttttttttaactttccctccacacgcatctacgtgaagaatcaagaaagtggtcataggtattcccccacaagtgaagtaatataaattccagaccaaacccataagaaaaaaaaaaaggaatcattcgagccacacccacaatggattcaaataaagagagaatcgaggacttggcttctttgttcttgccagatcttttacgcccttcaacagtcaattcgtcaccttcaaatgctactagtccccatgcatctaaatctgaagactacgatatgggttcagtatttttgaaagactcaaaacttaccaacgctccgagcaattcaggattaccaagcagttttcaagagtttttaagtaataacaattttatcaatagatcaaacgatatggaagatggccaagaagaaagatcacagcaacaaccacaacagtattcacatcattataatgggtatatgccttttgagatgaacccatctgtatcacaacacatgatgagttacaacccaggtatgcaactgatgttccaacaacccaataatccccagcaacaaagttttctttcttcaacacttcctggacaacaacaaatggtgtctaacttcccaagaactggttcttatgcaaaatatcctggtgagtatgatgaatggtttgccagtacgaatagtagcattaccaatttaaatggtctatatcagcaacagttgttgcagctgcaagctttacaacaaccacagccaatgccacaaccacagaatggagccaaggatacacgtatggatacccaaagtggctcatcgactccaaagaggaaaaaagccaagagtagaaaacgagacgtaaaaaatctcgaggttcaaatcgactacaaacctagtaaattgaaacgattacttgatctcaaacaatctggcgcaacgtcttcaaatgattataaaataatagacaaggacaataatgaagtcactattgatttcaacgggtttttaaatggaagatttctcaccaacgatattgataataataattacatcttcactcagaatgaatcacagcgtggcgaagacgagcttcctaaaactgttgtcaacaaaaaagagaatcctaaagttgtgtcatgctatcgacgcaactatattcaaatatcagccaatatgagcattcgtggatttaaaaatgattcaaagttgttgaaactacagaccagcgagtatggctacactacaactagagtgattaaatatttcaaaatagaggtgctggcagcagctaacaattcaaacagcaaaggagttcctataattatcaagaacgagaacaaggatatagagagggaacgggaaaaagaagcaagaaaaatgagtttaccttccaattatgaatacaaagctgacaatgttctgcctacatatatcaacaccaacgaatatgtcataatattgaacgatgaacctatcagcaatggaatgattgacaagtactttgtggtgaaaaaattacaatttaaaaatgcaacaccaaacaacgggagtttgacgtttcaaaactattaccatttaaaagccaagttgagttgtattgttgctgatatttattacgatgattacgatattgatgtggccaatgatggcggtggtggtaacaatgaaatattgttggctgaactagttagtgagcccataattgttcgtggtcgtaacccaagcttctacgctgaaagaaacgatattctaatcaagggtcgatctgctagttccaagtcatcattcaagatagcaggacaatcgtctgagctcaaatttagagctgcagcagatgcagaggaagatgaagacgatggattcgttgtggagcatgaagacgagccagaagatgaagatgaccatcacaaccacagtgaaggtgatgtggctggccaggaagaccaactaccccacacccaaaatggaactggagaatcaccaatctcatctggcgacgacgacgaaaaagacaatacacaaataccgccattatcatattcgaccaaccagttggctttggatgtcaagtctgtcgacaaatacaaatattatcccatcaacagcgtatattacctcccaccgataaacgtggtatattttccccacagagctcatcaatcacaaaaagacgatcaagaacagacttcaattgttcaagaaaacagaaagagttccaatgtttatttcaaatgatacagaaaaagacaatgccgagacagttatatttttaatgtagttatttatatttttttatttcttttatttgtaatatttagtatcttacatgtagatgtctgttacatttgtatgttcattacttgctatacaatacacacatttatttttgattcataagacgcctattccttaatcaatatggaggcagcacccataccactaccgatacacatagaagtcaaaccaatttcacctggtttcaacaatctcaagatagtggcatattgtctggcaccagtacaaccaagaggatgacctaaagcgatggcaccaccgtttatgttcaatttttcttctggaacattacattgttcagctgagtaaagacattgagcagcaaaagcttcgttgatttcaaacacatcaacgtcatccacagtca

SEQ ID NO: 5 - DNA, 26 bp, Artificial Sequence
  Description of Artificial Sequence Synthetic primer
tttcccgggggatcctcgttactcaa

SEQ ID NO: 6 - DNA, 33 bp, Artificial Sequence
  Description of Artificial Sequence Synthetic primer
cccaagcttgcataatttttttctttttgacct

SEQ ID NO: 7 - DNA, 28 bp, Artificial Sequence
  Description of Artificial Sequence Synthetic primer
ttttcccgggcagcagtttagaagcaat

SEQ ID NO: 8 - DNA, 27 bp, Artificial Sequence
  Description of Artificial Sequence Synthetic primer
cccccccgggtgatttgtcttaacatt

SEQ ID NO: 9 - DNA, 28 bp, Artificial Sequence
  Description of Artificial Sequence Synthetic primer
cgggatccttgtggcgattttcactttc

SEQ ID NO: 10 - DNA, 26 bp, Artificial Sequence
  Description of Artificial Sequence Synthetic primer
ccggatcctcaatgggggtggattga

SEQ ID NO: 11 - DNA, 30 bp, Artificial Sequence
  Description of Artificial Sequence Synthetic primer
aacccaagcttgcattgtgaagttctatgt

SEQ ID NO: 12 - DNA, 27 bp, Artificial Sequence
  Description of Artificial Sequence Synthetic primer
cgcggatccaccaattaatcacaacgg

SEQ ID NO: 13 - DNA, 27 bp, Artificial Sequence
  Description of Artificial Sequence Synthetic primer
gcgggatcctcatgtaaccttgcaatc

SEQ ID NO: 14 - DNA, 29 bp, Artificial Sequence
  Description of Artificial Sequence Synthetic primer
cgcggatcccttagacttacttatatccg

SEQ ID NO: 15 - DNA, 28 bp, Artificial Sequence
  Description of Artificial Sequence Synthetic primer
cgcggatccggcttgctaaacattatca

SEQ ID NO: 16 - DNA, 27 bp, Artificial Sequence
  Description of Artificial Sequence Synthetic primer
cgcggatccagagaatccagaaaagag

SEQ ID NO: 17 - DNA, 25 bp, Artificial Sequence
  Description of Artificial Sequence Synthetic oligonucleotide
tttccgggcagcatttagaagcaat

SEQ ID NO: 18 - DNA, 27 bp, Artificial Sequence
  Description of Artificial Sequence Synthetic oligonucleotide
cccccccgggtgatttgtcttaacatt

(19) United States
(12) Patent Application Publication
(10) Pub. No.: US 2005/0192240 A1
(43) Pub. Date: Sep. 1, 2005

(54) Methods and compositions relating to expression factors

(76) Inventors: Yun-Liang Yang, Sijhih City (TW); Chia-Geun Chen, Jiouru Township (TW); Hsiu-Jung Lo, Sijhih City (TW)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/030,965

(22) Filed: Jan. 10, 2005

Related U.S. Application Data
(60) Provisional application No. 60/535,000, filed on Jan. 9, 2004.

(51) Int. Cl. A61K 48/00

(52) U.S. Cl. 514/44

(57) ABSTRACT

The invention describes an expression factor and methods for inhibiting the growth of cells, for enhancing the activity of a drug, and for inhibiting the virulence of microbes. Methods of screening for expression factor inhibitors are also described. The compositions comprise at least one expression factor inhibitor and may further comprise at least one drug.

### RELATED APPLICATION

This application claims the benefit of U.S. Provisional Application No. 60/535,000, filed Jan. 9, 2004, which is hereby incorporated by reference in its entirety.

### FIELD OF THE INVENTION

The present invention relates to expression factors. Modulation of the expression and/or activity of expression factors may modulate, for example, drug efflux, virulence, and/or growth of cells. Expression factors include, but are not limited to, transcription factors, translation factors, repressors, and efflux pump expression factors. Expression factors may include one or more mid-sporulation elements (MSE) in its polynucleotide or may bind to a MSE in a target polynucleotide to regulate expression of itself or the target polynucleotide. Drug efflux, virulence, and/or growth of cells may be modulated by modulating the binding of an expression factor to a MSE, by modulating the expression of an efflux pump, or by modulating the expression or activity of the expression factor itself.

### BACKGROUND OF THE INVENTION

Due to wide use of antimicrobial agents for both treatment (and in many cases, treatment continued for a prolonged period) and prophylaxis, drug resistance has become a common cause of therapy failure (see e.g., Rex et al., Antimicrob. Agents and Chemother. 39:1-8, 1995; Vanden Bossche et al., Trends Microbiol. 2:393-400, 1994). One common mechanism of drug resistance is the modification or mutation of the target enzyme and/or other enzymes in the same biochemical pathway. For example, a point mutation in the C. albicans ERG11 gene was identified in an azole-resistant clinical isolate (White, Antimicrob. Agents Chemother. 41:1488-1494, 1997). Mutations in the ERG3 gene have also been reported as being responsible for resistance in at least two clinical isolates of C. albicans (Kelly et al., FEBS Lett. 400:80-82, 1997).

However, many of the cases of antimicrobial drug resistance appear to originate from decreased accumulation of the drug inside resistant cells (see, e.g., Vanden Bossche et al., Trends Microbiol. 2:393-400, 1994; Odds, J. Antimicrob. Chemother. 31: 463-471, 1993). Decreased drug accumulation may be caused by either decreased uptake of the drug and/or increased efflux of the drug from the cell.

Active efflux is associated with membrane transporter proteins, also known as efflux pumps. These pumps are ubiquitous from bacteria to mammals (for a review, see e.g., Higgins, Annu. Rev. Cell Biol. 8:67-113, 1992). Eukaryotic cells contain two types of efflux pumps that are known to contribute to drug resistance: the ATP binding cassette (ABC) transporters and major facilitators (Marger and Saier, Jr. Trends Biochem. Sci. 18:13-20, 1993; Michaelis and Berkower, Cold Spring Harbor Symp. Quant. Biol. 60:291-309, 1995). The ABC transporters utilize the energy from ATP hydrolysis for activity, and the major facilitators use the energy from proton transfer.

Overexpression of efflux pumps has been shown to contribute to drug resistance of infectious agents (Karababa et al., Antimicrob. Agents Chemother. 48:3064-3079, 2004; Marger and Saier, Jr., Trends Biochem. Sci. 18:13-20, 1993; Michaelis and Berkower, Cold Spring Harb. Symp. Quant. Biol. 60:291-307, 1995). For example, the CDR1 gene from the fungus Candida albicans encodes an ATP binding cassette efflux pump (Prasad et al., Curr. Genet. 27:320-329, 1995). Mutations on CDR1 resulted in a decreased expression of the efflux pump and increased susceptibility of C. albicans to azole drugs (Sanglard et al., Antimicrob. Agents Chemother. 40:2300-2305, 1996), consistent with the observation that overexpression of CDR1 contributes to the drug resistance of clinical isolates of C. albicans (Lopez-Ribot et al., Antimicrob. Agents Chemother. 42:2932-2937, 1998; Yang and Lo, J Microbiol Immunol Infect 34:79-86, 2001).

Expression of efflux pumps appears to be regulated by cis and trans-regulatory factors. For example, the AP-1 site and Drug Responsive Element (DRE) of the CDR1 promoter have thus far been reported as cis-regulatory elements (de Micheli et al., Mol. Microbiol. 43:1197-1214, 2002; Puri et al., FEMS Microbiol. Lett. 180:213-219, 1999), while the existence of trans-regulatory factors of CDR1 has been suggested (Puri et al., FEMS Microbiol. Lett. 180:213-219, 1999).

Indeed, a better understanding of the molecular mechanism and the gene network regulating the expression of efflux pumps would enhance future drug design and development.

### THE INVENTION

The present invention is based on the discovery by the inventors of an expression factor that regulates expression of efflux pumps. Additionally, the inventors discovered that the expression factor may be involved in a signal transduction pathway that does not involve efflux pumps. For example, an expression factor of the invention may include one or more mid-sporulation elements (MSE) in its polynucleotide or bind to a MSE in a target polynucleotide to regulate expression of its own polynucleotide or the target polynucleotide. Thus, modulation of the expression and/or activity of an expression factor of the invention may have a variety of effects on a cell, including inhibiting drug efflux, virulence, and/or growth of cells. Expression factors include, but are not limited to, transcription factors, translation factors, repressors, and efflux pump expression factors.

One aspect of the invention provides a method of inhibiting the growth of a cell by inhibiting the expression or activity of at least one expression factor in the cell, and may further comprise contacting the cell with at least one drug. In another aspect, the invention provides a method of inhibiting the growth of a cell by contacting the cell with at least one expression factor inhibitor and may further comprise contacting the cell with at least one drug. In an embodiment of the invention, the expression factor may be an efflux pump expression factor and the expression factor inhibitor may be an efflux pump expression factor inhibitor. Thus, the present invention also provides a method of enhancing the activity of a drug by contacting the cell with at least one efflux pump expression factor inhibitor, and at least one drug.

Another aspect of the invention provides a method of inhibiting the growth of a cell by inhibiting the expression of at least one efflux pump polynucleotide in the cell, and may further comprise contacting the cell with at least one drug. The present invention also provides a method of enhancing the activity of a drug by inhibiting the expression of at least one efflux pump polynucleotide in the cell, and contacting the cell with at least one drug.

Another method of inhibiting the growth of a cell includes inhibiting the binding of at least one expression factor to a mid-sporulation element (MSE). The method may further comprise contacting the cell with at least one drug.

The invention also provides a method of inhibiting virulence of a cell by inhibiting the expression or activity of at least one expression factor. The method may comprise contacting the cell with at least one expression factor inhibitor and may further comprise contacting the cell with at least one drug.

The present invention also provides a method for treating a patient with a disease, disorder, or infection. The method comprises administering to the patient an effective amount of at least one expression factor inhibitor and may further comprise administering an effective amount of at least one drug. Another aspect of the invention provides a pharmaceutical composition comprising at least one expression factor inhibitor and may further comprise at least one drug. The pharmaceutical composition may also further comprise a pharmaceutically acceptable carrier.

The invention also provides a composition for inhibiting the growth of a cell, comprising at least one expression factor inhibitor and may further comprise at least one drug. The present invention also provides a composition for inhibiting the virulence of a microbial cell, comprising at least one expression factor inhibitor and may further comprise at least one antimicrobial agent.

The present invention further provides a method for screening for a polynucleotide encoding an efflux pump expression factor. The method comprises introducing a reporter gene under the control of the efflux pump promoter into a host cell, introducing a candidate efflux pump expression factor polynucleotide into the host cell, and detecting the expression of the reporter gene. The method may be used to screen a genomic or cDNA library. for an efflux pump expression factor polynucleotide.

### BRIEF DESCRIPTION OF THE DRAWINGS

FIG. 1 FIGS. 1A and 1B show a comparison of the amino acid sequences of CaNdt80p (SEQ ID NO: 1) and Ndt80p (SEQ ID NO: 2).

FIG. 2 FIGS. 2A and 2B show the nucleotide sequence of CaNDT80 (SEQ ID NO: 3). The perfect MSE is shown at 572 bp upstream of the initiation codon (+1).

FIG. 3 FIGS. 3A and 3B show the nucleotide sequence of REP1 (SEQ ID NO: 4).

FIG. 4 FIG. 4 illustrates the integration of the LOB43 plasmid harboring the CDR1 promoter (CDR1p)-lacZ fusion, into the ADE3 locus in S. cerevisiae. Horizontal arrows indicate the direction of transcription from the CDR1 promoter.

FIG. 5 FIG. 5 is a bar graph showing the relative β-galactosidase (β-Gal) activities in the SLO1 S. cerevisiae strain transformed with (A) the control 2μ-URA3 vector (designated as strain SLO2); (B) 2μ-URA3-CaNDT80 (LOB44) plasmid (designated as strain SLO3); and in the SLO3 S. cerevisiae strain (C) in which the LOB44 plasmid was removed (designated as strain SLO4) and (D) retransformed with the LOB44 plasmid after removal of the LOB44 plasmid (designated as strain SLO5).

FIG. 6 FIG. 6 shows the results of the Etest (AB BIODISK, Solna, Sweden) for susceptibility of various strains to the antifungal drugs fluconazole (FL) (bottom panels) and ketoconazole (KE) (top panels). The strains tested were the Ndt80/NDT80 wild type strain of S. cerevisiae (NDT80/NDT80); S. cerevisiae with a homozygous ndt80/ndt80 mutation transformed with the 2μ-URA3 control vector (ndt80/ndt80 2μ vector); the ndt80/ndt80 mutant strain transformed with the 2μ-URA3-NDT80 plasmid (ndt80/ndt80 2μ NDT80); and the ndt80/ndt80 mutant strain transformed with the 2μ-URA3-CaNDT80 plasmid (ndt80/ndt80 2μ CaNDT80).

FIG. 7 FIG. 7 shows the results of the Etest (AB BIODISK, Solna, Sweden) for susceptibility of S. cerevisiae transformed with 2μ control vector or with 2μ LOB44 to the antifungal drugs fluconazole (FL) (FIGS. 7A and 7B, respectively) and ketoconazole (KE) (FIGS. 7C and 7D, respectively).

FIG. 8 FIG. 8 depicts the construction of a null mutation in CaNDT80. The two copies of the CaNDT80 gene (open box) were sequentially replaced by the GFP-ARG4 construct (solid box) and the URA3-dp/200-based cassette (hatch box).

FIG. 9 FIG. 9 shows reverse transcriptase PCR products amplified from various C. albicans strains treated with (lanes 11-19) and without (lanes 2-10) miconazole (Sigma) and electrophoresed on an agarose gel. Lane 1: molecular weight standard (1 kb plus ladder from Invitrogen); lanes 2-4 and 11-13: the homozygous mutant strain Candt80/Candt80 (YLO133); lanes 5-7 and 14-16: strain Candt80/Candt80::CaNDT80 (YLO137); lanes 8-10 and 17-19: wild type strain CaNDT80/CaNDT80 (SC5314). Lanes 2, 5, 8, 11, 14, and 17 were tested for TEF3 mRNA (internal control); lanes 3, 6, 9, 12, 15, and 18, were tested for CDR1 mRNA; and lanes 4, 7, 10, 13, 16, and 19 were tested for CaNDT80 mRNA.

FIG. 10 FIG. 10 shows the results of the Etest (AB BIODISK, Solna, Sweden) for susceptibility of various strains to the antifungal drugs amphotericin B (AP), 5-fluorocytosine (FC), fluconazole (FL), itraconazole (IT), and ketoconazole (KE). The strains tested were the C. albicans (A) wild type strain CaNDT80/CaNDT80 (SC5314), (B) homozygous mutant strain Candt80/Candt80 (YLO133), and (C) the homozygous mutant strain complemented with a wild type CaNDT80 (YLO137).

FIG. 11 FIG. 11 shows photographs of wild type C. albican strain SC5314 (wt), cdr1/cdr1 homozygous mutant strain DSY448 (cdr1/cdr1), Candt80/Candt80 homozygous mutant strain YLO133 (Candt80/Candt80), and Candt80/Candt80 rescued strain YLO137 (Candt80/Candt80::CaNDT80) cells grown on agar plates in the absence of drug (A), in the presence of 25 μg/ml fluconazole (B), and in the presence of 1 μg/ml voriconazole (C).

FIG. 12 FIG. 12 shows photographs of C. albicans strains CaNDT80/CaNDT80 (wild type), Candt80/Candt80 (homozygous mutant), and a rescued Candt80/Candt80::CaNDT80 strain in a germ tube formation assay.

FIG. 13 FIG. 13 is a graph showing the virulence of C. albicans in a mouse model injected with the wild type strain, CaNDT80/CaNDT80, and the homozygous mutant, Candt80/Candt80.

### DESCRIPTION OF THE INVENTION

The present invention provides a method for inhibiting the growth of a cell by inhibiting the expression or activity of at least one expression factor in a cell. Expression factors include, but are not limited to, transcription factors, translation factors, repressors, and efflux pump expression factors. The present invention also provides a method for inhibiting the growth of a cell by inhibiting the expression of at least one efflux pump polynucleotide in a cell, and contacting the cell with at least one drug.

The expression factor may also affect virulence of the cell and, therefore, inhibition of the expression or activity of the expression factor may inhibit virulence of the cell. Accordingly, the present invention provides a method of inhibiting the virulence of a cell by inhibiting the expression or activity of at least one expression factor of the cell. Virulence of a cell may be inhibited by contacting the cell with at least one expression factor inhibitor and may further comprise contacting the cell with at least one drug.

In another aspect, the expression factor may affect the growth of the cell by affecting a mid-sporulation element (MSE). The MSE may regulate the expression of an expression factor polynucleotide and/or a target polynucleotide of the expression factor. Thus, the present invention provides a method of inhibiting the growth of a cell by inhibiting the binding of an expression factor to a MSE and may further comprise contacting the cell with at least one drug.

In an embodiment, the cell is a plant cell, a bacterial cell, a yeast cell, or a mammalian cell. In yet another embodiment, the yeast cell is a fungal cell and may be selected from the Candida species, Aspergillus species, and the Cryptococcus species. The Candida species may be selected from C. albicans, C. krusei, C. glabrata, and C. tropicalis. The Aspergillus species may be selected from Aspergillus fumigatus and Aspergillus niger. The Cryptococcus species may be selected from Cryptococcus neoformans. Other fungal cells are known in the art. Mammalian cells include cells of rodent, human, non-human primates, equines, canines, felines, bovines, porcines, ovines, lagomorphs, and the like.

In another embodiment, the efflux pump polynucleotide is selected from CDR1, CDR2, and MDR1, or homologs or variants thereof. The expression of the efflux pump may be inhibited by inhibiting the transcription and/or the translation of the efflux pump polynucleotide. In one embodiment, expression of the efflux pump polynucleotide is modulated by an efflux pump expression factor. One embodiment of the efflux pump expression factor is CaNdt80p. In another embodiment, the efflux pump expression factor is the Regulator of Efflux Pump 1 (REP 1). Accordingly, the present invention also provides a method of inhibiting the growth of a cell by inhibiting the expression or activity of at least one efflux pump expression factor. Another aspect of the invention comprises contacting the cell with at least one efflux pump expression factor inhibitor and may further comprise contacting the cell with at least one drug.

The embodiments described and the terminology used herein are for the purpose of describing exemplary embodiments only, and are not intended to be limiting. The scope of the present invention is intended to encompass additional embodiments not specifically described herein, but that would be apparent to one skilled in the art upon reading the present disclosure and practicing the invention.

Where applicable, polynucleotide names have been italicized. “Polynucleotide,” “nucleotide,” and “nucleic acid” refer to DNA or RNA. Wild type polynucleotides have also been capitalized, whereas mutant polynucleotides have not been capitalized. Moreover, “Ca” of CaNDT80, for example, refers to the Candida albicans homolog of NDT80 in S. cerevisiae. The proteins have not been italicized and only the first letter of the protein name has been capitalized. Some proteins are succeeded with the letter “p” to indicate a protein. Where these rules are not followed, it will be apparent to one ordinary in the art whether the name indicates a wild type or mutant, or a polynucleotide or a protein.

The present invention relates to the modulation of an expression factor, or a homolog or variant thereof. “Modulates” or “modulation” refers to the increase or decrease in expression or activity of an expression factor. “Activity” refers to a function or set of activities performed by a molecule and may include, among others, the binding to target molecules and activation or inhibition of transcription and/or translation of itself or a target polynucleotide. “Expression” refers to translation and/or transcription of a polynucleotide or protein.

The terms “inhibition” or “inhibiting” refers to a decrease or cessation of any phenotypic change of a cell. In the context of cell growth, “inhibition” refers to a decrease or cessation in the rate of growth of the cell. For example, the microbial population may be decreased. Such inhibition may be monitored, for example, by the difference in turbidity of liquid cultures in the presence or absence of the inhibiting agent, or by the difference in size of inhibition zone for cultures on solid media in the presence or absence of the inhibiting agent, or by other methods well-known to those skilled in the art. “Inhibition” of expression of a polynucleotide refers to a decrease or cessation in transcription or translation of a polynucleotide. Inhibition of polynucleotide expression may be detected or monitored by the amount of mRNA or gene product produced in the presence or absence of the inhibiting agent, for example, by Northern blot and Western blot analyses.

The terms “activation” or “activating” refers to an increase or development of any phenotypic change of a cell. In the context of cell growth, “activation” refers to an increase in the rate of growth of the cell, or to the initiation of cell division. Such activation may be similarly monitored as described above. “Activation” of expression of a polynucleotide refers to an increase or initiation in transcription or translation of a polynucleotide. Activation of polynucleotide expression may also be monitored as described above.

The term “microbe” or “microbial cell” refers to very small organisms, which generally are only readily observable when viewed under a microscope or when aggregated. The term “microbe” or “microbial cell” includes, for example, bacteria, algae, fungi, and protozoans. The term “microbe” or “microbial cell” also includes to an organism in its polynucleate or single-celled state. Thus, an “antimicrobial agent” is a drug that inhibits the growth of a microbe or a microbial cell.

The term “drug” refers to compounds intended for use in diagnosis, cure, mitigation, treatment, or prevention of a disease or symptom in a patient and/or to compounds (other than food) intended to affect the structure or any function of the body or a cell. In an embodiment of the present invention, the drug acts to inhibit cell growth.

An expression factor may include, but is not limited to, a transcription factor, translation factor, and repressor. In a specific embodiment, the expression factor is an efflux pump expression factor. As used herein, “efflux pump” refers to a protein assembly which exports substrate molecules from the cytoplasm or periplasm of a cell, in an energy dependent fashion. In an embodiment of the invention, the efflux pump is involved in drug resistance by cells. Bacterial efflux pumps involved in drug resistance include QacA, NorA (Bmr), Smr, AcrAB, and MexAB-OprM (Lewis, Trends Biochem. Sci. 19:119-123, 1994; Nikaido, Science 264:382-388, 1994; Poole et al., J. Bacteriol. 175:7363-7372, 1993; Okusu et al., J. Bacteriol. 178:306-308, 1996; Nikaido, J. Bacteriol. 178:5853-5859, 1996). Eukaryotic efflux pumps include the ATP binding cassette (ABC) transporters and the major facilitators (MF) (White et al., Clinical Microbiol. Reviews 11:382-402, 1998) and are also found in humans (see, e.g., Litman et al., Cell Mol Life Sci. 58:931-59, 2001). In S. cerevisiae, at least thirty ABC transporters have been identified and are grouped into six families, based on their sequence similarities. The PDR5, MRP/CFTR, and MDR families contain members that are known to cause drug resistance in a variety of systems. Ten members of the PDR5 family have been described in C. albicans, and these genes are named CDR. In particular, the CDR1 and CDR2 efflux pumps are known to participate in resistance against azole compounds (Prasad et al., Curr. Genet. 27:320-329, 1995; Fling et al., Mol. Molec. Genet. 227:318-329, 1991; Sanglard et al., Antimicrob. Agents Chemother. 39:2378-2386, 1995; Sanglard et al., Antimicrob. Agents and Chemother. 40:2300-2305, 1996; Sanglard et al., Microbiology 143:405-416, 1997). Members of the MF family that participate in drug resistance in C. albicans include the MDR1 (or CaMDR1; formerly BENr) and FLU1 (Fling et al., Mol. Molec. Genet. 227:318-329, 1991; Calabrese et al., Microbiology 146:2743-2754, 2000).

The present invention also relates to homologs or variants of efflux pumps. As used herein, “homolog” refers to structures or processes in different organisms that show a fundamental similarity. For example, a homolog of CDR1 may play a functional role similar to that of CDR1, i.e., as an efflux pump. A homolog of the CDR1 polynucleotide may have a primary or secondary structure similar to the CDR1 polynucleotide. A homolog of the CDR1 polynucleotide may also express gene products having homologous structures with the CDR1 polynucleotide product.

As used herein, “variant” of an efflux pump refers to a naturally-occurring or synthetically produced amino acid sequence substantially identical to that of the efflux pump, but which has an amino acid sequence different from that of the efflux pump because of one or more deletions, substitutions, or insertions. Similarly, a variant of an efflux pump polynucleotide refers to a naturally-occurring or synthetically produced nucleotide sequence substantially identical to that of the efflux pump polynucleotide, but which has a nucleotide sequence different from that of the efflux pump polynucleotide because of one or more deletions, substitutions, or insertions. A variant of an efflux pump (or a product of an efflux pump polynucleotide variant) retains efflux pump activity or has enhanced efflux pump activity compared with the efflux pump.

Sequence “similarity” and/or “identity” are used herein to describe the degree of relatedness between two polynucleotides or polypeptide sequences. In general, “identity” means the exact match-up of two or more nucleotide sequences or two or more amino acid sequences, where the nucleotide or amino acids being compared are the same. Also, in general, “similarity” means the exact match-up of two or more nucleotide sequences or two or more amino acid sequences, where the nucleotide or amino acids being compared are either the same or possess similar chemical and/or physical properties. The percent identity or similarity can be determined, for example, by comparing sequence information using the GAP computer program, version 6.0 described by Devereux et al. (Nucl. Acids Res. 12:387, 1984) and available from the University of Wisconsin Genetics Computer Group (UWGCG). The GAP program utilizes the alignment method of Needleman and Wunsch (J. Mol. Biol. 48:443, 1970), as revised by Smith and Waterman (Adv. Appl. Math 2:482, 1981). Other programs for calculating identity and similarity between two sequences are known in the art.

For purposes of the invention, a homolog or variant of an efflux pump or efflux pump polynucleotide may exhibit at least about 20% nucleotide or amino acid identity with the efflux pump, at least about 30% nucleotide or amino acid identity, or at least about 40% nucleotide or amino acid identity, although the invention certainly encompasses sequences that exhibit at least about 50%, 60%, 70%, 80%, 90%, 95%, and 98% nucleotide or amino acid identity with the efflux pump. Naturally-occurring homologs and variants are encompassed by the invention. The present invention also relates to homologs or variants of expression factors.

A “DNA binding domain” refers to a region in a protein, polypeptide, peptide, or polynucleotide that binds to a specific DNA sequence. For example, the DNA binding domain of Ndt80p has been identified (Montano et al., PNAS 99:14041-6, 2002). It comprises a single 32-kDa domain with a beta-sandwich core elaborated with seven additional beta-sheets and three short alpha-helices. As described below, this DNA binding domain of Ndt80p has been found to be homologous to a number of proteins.

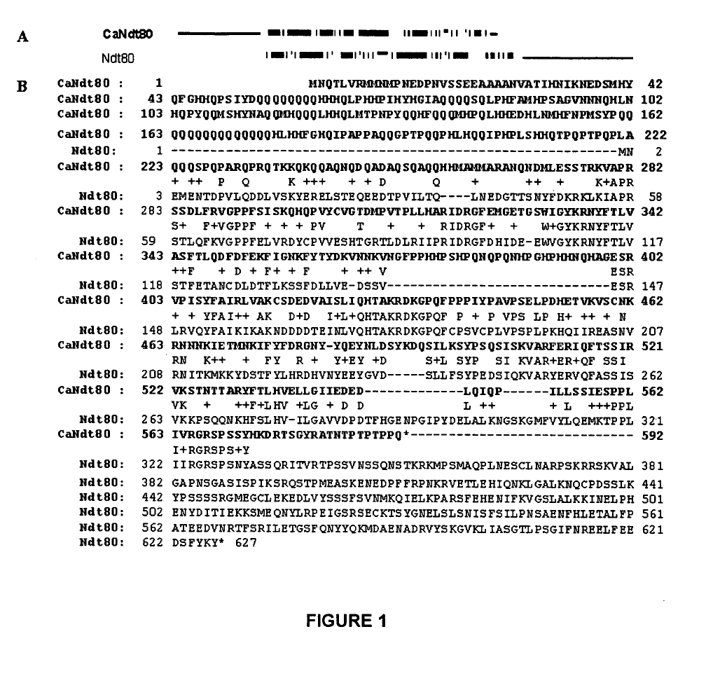

CaNdt80p is a C. albicans homolog of the S. cerevisiae Ndt80p, which is a meiosis specific transcription factor in S. cerevisiae (Chu et al., Science 282:699-705, 1998; Chu and Herskowitz, Mol. Cell 1:685-696, 1998). The amino acid sequences of CaNdt80p and Ndt80p are compared in FIG. 1. CaNdt80p is 592 amino acid in length while Ndt80p is 672 amino acids in length. In Ndt80p, residues 1 to 330 have been shown to be important for DNA binding. Two different domains important for DNA binding have been identified in this region. Residues 1 to 58 are involved in sequence-specific recognition and residues 59 to 330 contain a DNA binding domain. Residues 223 to 572 of CaNdt80p share 37.6% identity and 57.9% similarity (thick gray bars in FIG. 1A) with residues 3 to 330 of Ndt80p, suggesting that this region in CaNdt80p is also involved in DNA binding. There is no similarity between the N-terminal region of CaNdt80p and the C-terminal of Ndt80p (thin black lines in FIG. 1A).

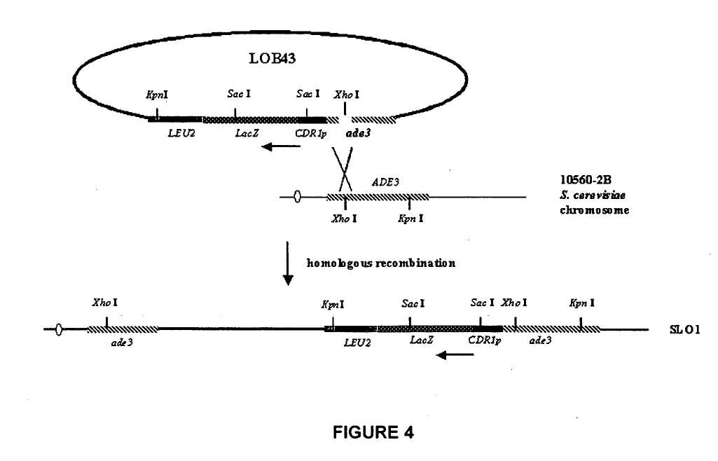

Because of the similarities between CaNdt80p and Ndt80p, analysis of Ndt80p may help elucidate the function of CaNdt80p. Ndt80p activates its targets by binding through its DNA binding domain to a mid-sporulation element consensus site (MSE), gNCRCAAAA/T, in the target polynucleotide (Chu and Herskowitz, Mol. Cell 1:685-696, 1998; Lamoureux et al., EMBO J. 21:5721-5732, 2002). The NDT80 polynucleotide itself also contains MSE sequences and it has been shown that Ndt80p induces its own synthesis, presumably at the transcriptional level through its MSE sequence (Lamoureux et al., EMBO J. 21:5721-5732, 2002). The expression of NDT80 is repressed in S. cerevisiae during vegetative growth (Xu et al., Mol. Cell Biol. 15:6572-6581, 1995; Pak and Segall, Mol. Cell Biol. 22:6417-6429, 2002), and neither overexpression of NDT80 nor mutations on NDT80 in S. cerevisiae alter drug susceptibility (see FIGS. 4B and 4C).

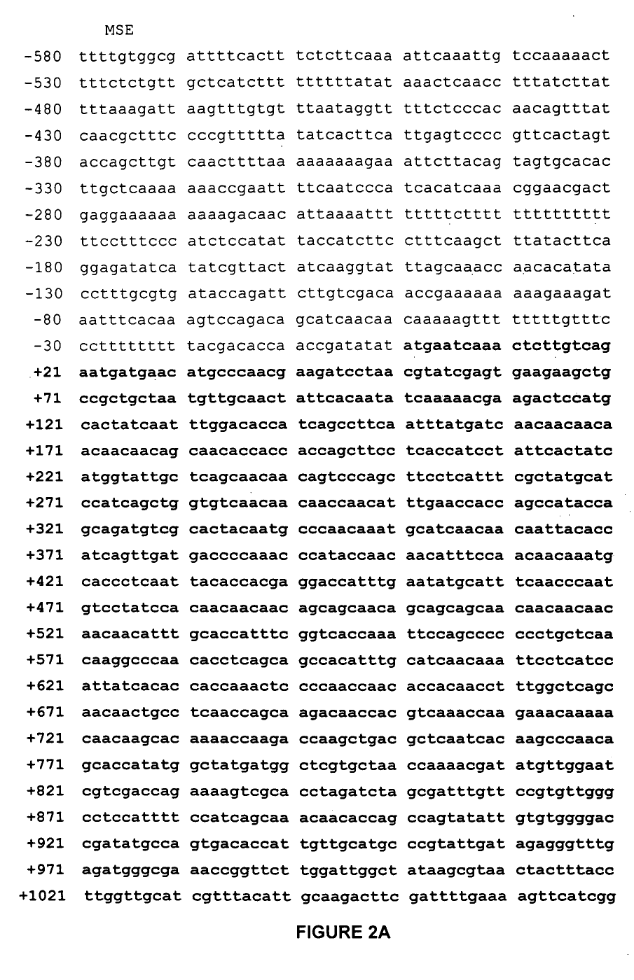

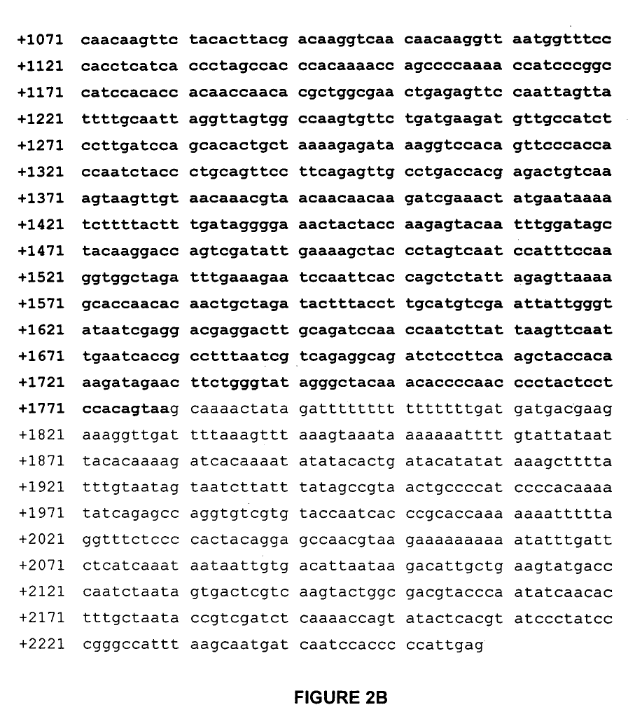

Interestingly, overexpression of the CaNDT80 polynucleotide reduced drug susceptibility and mutation of the CaNDT80 polynucleotide increased drug susceptibility by modulating the expression of the CDR1 efflux pump polynucleotide. Indeed, the promoter of CDR1 contains three potential MSEs (CRCAAA) located at 270 bp, 438 bp, and 835 bp from the translation initiating ATG. CaNDT80 also contains one perfect MSE at 572 bp upstream of the translation initiation codon of CaNDT80 and three potential MSEs (CRCAAA) at 70 bp, 122 bp, and 461 bp upstream of ATG. The nucleotide sequence of CaNDT80 is depicted in FIG. 2.

In addition to CaNdt80p and Ndt80p, several proteins from higher eukaryotes including Neurospora crassa, Dictyostelium discoideum, Caenorhabditis elegans, Drosophila melanogaster, and humans also contain sequences homologous to the DNA binding domain of Ndt80p (Montano et al., Proc. Natl. Acad. Sci. U.S.A. 99:14041-14046, 2002). Additionally, C11orf9, a human transcription factor with a similar DNA binding domain to Ndt80p, has been found to be highly expressed in invasive or metastatic tumors (Kiemer et al., Oncogene 20:6679-6688, 2001). Thus, inhibition of the activity of transcription factors that bind to MSEs may reduce or inhibit cancer cell growth. Moreover, because many metastatic tumors are more resistant to chemotherapeutic drugs than their primary cell counterparts, and because many drug resistant tumors are more invasive than non-resistant parental cells (Liang et al., Curr. Cancer Drug Targets. 2:257-277, 2002), inhibition of transcription factors regulating the expression of efflux pumps in human cells may also enhance the activity of chemotherapeutic drugs.

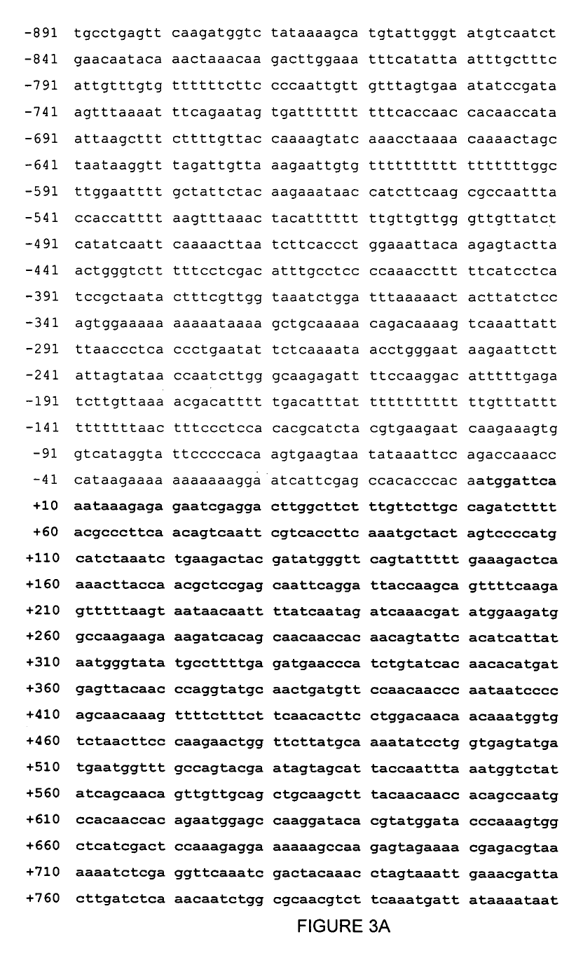

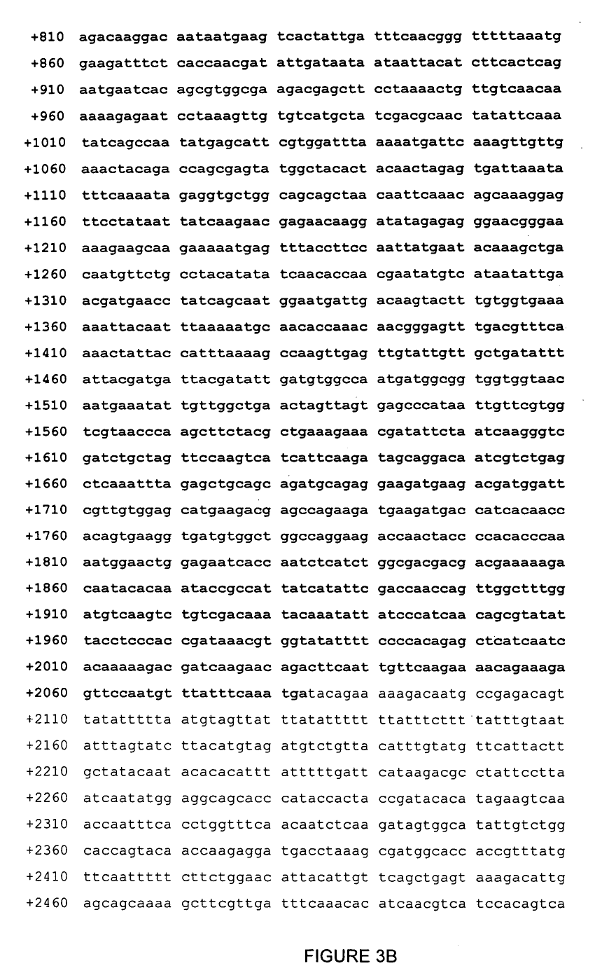

Thus, the efflux pump expression factor of the invention may be capable of binding to a midsporulation element (MSE) in a target polynucleotide. The “efflux pump expression factor” refers to a transcription factor, a repressor, or a translation factor that modulates the expression of an efflux pump polynucleotide. In an embodiment of the invention, the efflux pump expression factor is Candt80p. In another embodiment, the efflux pump expression factor is a novel REP (Regulator of Efflux Pump) 1. The C. albicans REP1 polynucleotide encodes a putative 693 amino acid protein. Overexpression of the REP1 polynucleotide increased gene expression from the CDR1 and MDR1 promoters. The nucleotide sequence of REP1 is depicted in FIG. 3.

A patient is hereby defined as any person or non-human animal in need of treatment for a disease, disorder, or infection, or to any subject for whom treatment may be beneficial, including humans and non-human animals. Such non-human animals to be treated include all domesticated and feral vertebrates. In an embodiment of the present invention, subjects for whom treatment may be beneficial include subjects undergoing drug therapy and subjects with cancer.

The present invention encompasses a method of inhibiting the growth of a cell by inhibiting the expression or activity of at least one expression factor in the cell, and may further comprise contacting the cell with at least one drug.

The present invention further relates to a method of enhancing the activity of a drug in a cell by inhibiting the expression or activity of at least one expression factor in the cell, and may further comprise contacting the cell with the drug. “Enhancing” or “enhance” refers to the increase in the effect that the drug has on the cell. The effect may be detected by a decrease or cessation in the rate of growth of the cell or by a decrease or cessation in transcription or translation of a polynucleotide, as described above.

In an embodiment of the present invention, the invention encompasses a method of inhibiting the growth of a fungal cell by inhibiting the expression or activity of at least one expression factor in the fungal cell, and may further comprise contacting the fungal cell with at least one antifungal agent. In another embodiment, the invention encompasses a method of enhancing the activity of an antifungal agent in a fungal cell by inhibiting the expression or activity of at least one expression factor in the fungal cell, and may further comprise contacting the fungal cell with the antifungal agent.

The terms “fungus” and “fungi” refer to lower eukaryotic organisms as generally understood by those skilled in the art. The morphogenesis of fungus, (e.g., C. albicans), characterizes its change from a harmless communal to an infectious pathogen. C. albicans is capable of producing yeast cells, pseudohyphae, and true hyphae. As part of the normal flora, C. albicans grows as a budding yeast. The term “yeast,” which is included in the context of this invention, refers to a lower eukaryotic organism that has a single celled growth stage and is classified within the fungi, based on properties such as cell structure, reproductive mechanisms, nucleic acid sequence similarities or other characteristics commonly utilized for classifying organisms. The pseudophyphae and true hyphae forms reflect the multicellular, filamentous colonies and are produced only during infection of a host and are generally in a polynucleate vegetative stage.

Fungi include the classes Zygomycetes, Ascomycetes, Basidiomycetes, Deuteromycetes, and Oomycetes. The Candida species is a member of the class Ascomycetes. While Candida albicans is the most abundant and significant species, Candida tropicalis, Candida glabrata, Candida parapsilosis, Candida krusei, and Candida lusitaniae have been isolated as causative agents of Candida infections. A recent increase in infections due to non-albicans Candida spp., such as Candida glabrata and Candida krusei has been reported (Abi-Said et al., Clin. Infect. Dis. 24:1122-1128, 1997; Aisner et al., Am. J. Med. 61:23-28, 1976; Arif et al., J. Clin. Microbiol. 34:2205-2209, 1996). Other fungi species include Aspergillus spp. and Cryptococcus spp. Others are known in the art.

The term “antifungal agent” refers to a drug which inhibits the growth of a fungus. The activity of an antifungal agent may essentially stop fungal cell growth (but does not kill the fungus) or kill the fungal cell (and may stop growth before killing the fungus). In developing antifungal agents to combat infection by C. albicans and other fungi, scientists have exploited a necessary component of fungal walls. Unlike bacteria, but similar to other eukaryotes, fungal membranes contain sterols, which are essential for viability of nearly all fungi. They are important for the fluidity and integrity of the membrane and for the proper function of many membrane-bound enzymes, which are important for proper fungal cell growth and division (Joseph-Home and Hollomon, FEMS Microbiol. Lett. 149:141-149, 1997). The principle fungal sterols are ergosterol and zymosterol, while mammalian cell membranes contain cholesterol. Scientists have exploited differences in sterol composition for the development of antifungal agents, focusing on antifungal agents that complex with sterols of fungi but not with those of humans. The effect of these antifungal agents is to perforate fungal membranes while keeping human cell membranes intact.

Examples of such antifungal agents currently used are amphotericin B and nystatin, which are polyene macrolide antibiotics. These agents exhibit greater affinity for the ergosterol of fungal membranes than for the cholesterol of mammalian membranes. These drugs intercalate into the membranes, form a channel through which cellular components, especially potassium ions, leak and thereby destroy the proton gradient within the membrane (Vanden Bossche et al., Trends Microbiol. 2:393-400, 1994).

Other anti-fungal agents, such as allylamines (Ryder, N. S., Br. J. Dermatol. 126 Suppl 39:2-7, 1992), thiocarbamates, azoles, and morpholines, target enzymes involved in ergosterol biosynthesis. For example, azoles (e.g., fluconazole, itraconazole and ketoconazole) bind to and inhibit lanosterol demethylase, a cytochrome P-450 enzyme, which usually converts 14-α-methyl-sterols to ergosterol (Saag and Dismukes, Antimicrob. Agents Chemother. 32:1-8, 1988; Hitchcock, Biochem. Soc. Trans. 19:782-787, 1991).

Other antifungal agents used to fight against fungal infections include flucytosine (5-FC). 5-FC acts by inhibiting DNA and RNA synthesis. 5-FC is converted to 5-fluorouracil (5-FU) by cytosine deaminase inside the fungal cell. Cytosine deaminase has low activity in mammalian cells and therefore, 5-FC exhibits low toxicity in humans. 5-FU is converted into 5-fluorouridilic acid, which becomes phosphorylated (5-fluoro-UTP) and incorporated into RNA. Incorporation of 5-fluoro-UTP into RNA disrupts protein synthesis and inhibits fungal growth. 5-FU is also converted to 5-fluoro-dUTP, which is a potent inhibitor of thymidilate synthase, an essential enzyme for DNA synthesis (White et al., Clin. Microbiol. Rev. 11:382-402, 1998).

A review of anti-fungal agents is discussed in, for example, Odds, J. Antimicrob. Chemother. 31: 463-471, 1993, and Yang and Lo, J. Microbiol Immunol. Infect. 34:79-86, 2001. As used herein, “antifungal agent” refers to, but is not limited to, any one or more of the antifungal agents discussed. Other antifungal agents are known in the art.

The present invention also relates to virulence of a microbial cell. “Virulence” refers to the capacity of a pathogen to produce infectious forms of the pathogen and to infect a host cell. Virulence of a microbial cell may be inhibited by inhibiting the expression or activity of an expression factor. Thus, in the context of the present invention, “inhibiting virulence” refers to the decrease or cessation of the capacity of a microbial cell to infect a host cell and to produce infectious forms of the microbial cell.

In another embodiment of the invention, growth of a cell may be inhibited by contacting the cell with at least one efflux expression factor inhibitor. Another embodiment may further comprise contacting the cell with at least one drug. Virulence of a cell may also be inhibited by contacting the cell with at least one expression factor inhibitor, and the method may further comprise contacting the cell with at least one drug. In yet another embodiment, the activity of a drug may be enhanced by contacting the cell with at least one efflux expression factor inhibitor, and may further comprise contacting the cell with at least one drug. In a particular embodiment, the cell may be a fungal cell and thus, the drug may be an antifungal agent.

An “expression factor inhibitor” is a substance which interferes with the expression or activity of an expression factor. As a result, the expression factor inhibitor may interfere with, for example, the expression or activity of a target molecule of the expression factor, binding of the expression factor to its target molecule, or the export of a substrate or other compounds, such as antifungal agents, from the cell. Examples of expression factor inhibitors include antisense oligonucleotides that specifically hybridize with one or more nucleic acids encoding an expression factor, RNAi or interfering RNA, chemicals, and naturally-occurring, recombinant, or synthetic peptides, polypeptides, proteins, polysaccharides, small molecules and other compounds designed to reduce or inhibit the expression or activity of an expression factor. The expression factor inhibitor may inhibit binding of an expression factor to a mid-sporulation element (MSE) on its own polynucleotide, an efflux pump polynucleotide, or any other target polynucleotide. Thus, the present invention also provides a method of inhibiting growth of a cell by inhibiting the binding of an expression factor to a MSE.

In an embodiment of the invention, the expression factor inhibitor is an efflux pump expression factor inhibitor. An “efflux pump expression factor inhibitor” is a substance which interferes with the expression of an efflux pump polynucleotide by inhibiting the activity or expression of an efflux pump expression factor. As a result, the efflux pump expression factor inhibitor may interfere with the export of a substrate or other compounds, such as antifungal agents, from the cell.

An antisense oligonucleotide refers to a nucleic acid (RNA or DNA) that modulates the function of a nucleic acid molecule encoding a protein, ultimately modulating the amount of protein produced. Modulation of the amount of protein produced is accomplished by providing antisense oligonucleotides that specifically hybridize with one or more nucleic acids encoding the protein. As used herein, the terms “target nucleic acid” and “nucleic acid encoding a protein” encompass DNA encoding the protein, RNA (including pre-mRNA and mRNA) transcribed from such DNA, and also cDNA derived from such RNA. The specific hybridization of an antisense oligonucleotide with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. Uses of antisense oligonucleotides are known in the art. See, e.g., Holt et al., Mol. Cell Biol. 8:963-973, 1988; Anfossi et al., Proc. Natl. Acad. Sci. USA 86:3379-3383, 1989; Wickstrom et al., Proc. Nat. Acad. Sci. USA 85:1028-1032, 1988, Higgins et al., Proc. Nat. Acad. Sci. USA 90:9901-9905, 1993; Kitajima et al., Science 258:1792-1795, 1992; Li et al., Clin. Cancer Res. 8:3570-3578, 2002; Rijcken et al., Gut 51:529-535, 2002.

The overall effect of such interference with target nucleic acid function is modulation of the expression of the polynucleotide. In the context of the present invention, “modulation of the expression of the polynucleotide” means either an increase or a decrease in the expression of a polynucleotide. In an embodiment of the present invention, the antisense oligonucleotide may be designed to specifically hybridize to the mid-sporulation element (MSE) sequence of the expression factor polynucleotide or to the MSE of a target polynucleotide of an expression factor. In another embodiment of the invention, the antisense oligonucleotide is a ribozyme, which is an antisense RNA molecule that hybridizes to a target RNA and specifically cleaves the phosphodiester backbone of the target RNA. The ribozyme may catalyze the cleavage of its own RNA or other target RNA.

The antisense oligonucleotides used in this invention may be conveniently and routinely made through the well-known technique of solid phase synthesis. Equipment for such synthesis is sold by several vendors including Applied Biosystems (Foster City, Calif.). Any other means for such synthesis may also be employed; the actual synthesis of the oligonucleotides is well within the talents of those of ordinary skill in the art. It is also well known to use similar techniques to prepare other oligonucleotides such as the phosphorothioates and alkylated derivatives (Zon, Pharm. Res. 5:539-549, 1988). It is also well known to use similar techniques and commercially available modified amidites and controlled-pore glass (CPG) products such as biotin, fluorescein, acridine or psoralen-modified amidites and/or CPG (available from Glen Research, Sterling Va.) to synthesize fluorescently labeled, biotinylated or other modified oligonucleotides such as cholesterol-modified oligonucleotides.

The antisense oligonucleotides of the invention will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression. See Wagner et al., Nature Biotechnol. 14:840-844, 1996. In the context of this invention, it is understood that this encompasses naturally and non-naturally occurring oligomers as hereinbefore described.

Antisense molecules may also be produced by in vivo expression of all or a part of the target polynucleotide sequence in an appropriate vector, where the transcriptional initiation is oriented such that an antisense strand is produced as an RNA molecule. The RNA antisense sequence is complementary to the RNA of the targeted polynucleotide, and inhibits expression of the targeted polynucleotide products. Antisense molecules inhibit gene expression through various mechanisms, e.g. by reducing the amount of mRNA available for translation, through activation of RNAse H, or steric hindrance.

Alternatively, antisense molecules may be produced by in vitro transcription of all or part of the target polynucleotide sequence from an appropriate vector, as described above, such that an antisense strand is produced as an RNA molecule. Synthesis of single-stranded RNA in vitro has been facilitated by the development of plasmid vectors containing polycloning sites downstream from powerful promoters derived from the Salmonella typhimurium bacterophage SP6 (Green et al., Cell 32:681, 1983) or from the E. coli bacterophages T7 and T3 (Studier and Rosenberg, J. Mol. Bio. 153:503, 1981; Davanloo et al., Proc. Natl. Acad. Sci. USA 81:2035, 1984; Tabor and Richardson, Proc. Natl. Acad. Sci. USA 82:1074, 1985). The DNA-dependent RNA polymerases encoded by their respective bacteriophages specifically recognize their cognate promoters and do not use promoters recognized by other polymerases, such as other bacteriophage, bacterial or eukaryotic promoters present in a plasmid vector. Thus, when a linearized plasmid is incubated in vitro with the appropriate DNA-dependent RNA polymerase and the four rNTPs (ribonucleotide triphosphates), virtually all RNA synthesis is initiated at the select bacteriophage promoter. In vitro transcription may be performed by any of the methods known in the art. See Sambrook et al., Molecular Cloning: A Laboratory Manual, Vols 1-3 (2d ed. 1989), Cold Spring Harbor Laboratory Press; Milligan et al., Nucl. Acids Res. 15:8783, 1987; Milligan and Uhlenbeck, Meth. Enzymol. 180:51, 1989. Any of the commercially available kits may also be used, e.g., Riboprobe® In Vitro Transcription Systems from Promega (Madison, Wis.).

The term “RNAi” or “interfering RNA” refers to RNA that is partially or fully double-stranded similar to a portion of the target nucleic acid. When RNAi enters a cell, it triggers a cellular process that causes the degradation of not only the invading dsRNA molecule, but also single-stranded (ssRNAs) RNAs of identical sequences, including endogenous mRNAs. Thus, the overall effect of RNAi on target nucleic acid function is a decrease or inhibition of the expression of the target gene product, such as a protein or a ribozyme. For a review, see e.g., Nishikura, Cell 107:415-418, 2001; Hannon, Nature 418:244-251, 2002. For purposes of the invention, RNAi may be designed to target any portion of the target nucleic acid. For example, RNAi may target a ribozyme. In another embodiment, the target portion may comprise the mid-sporulation element (MSE) of an expression factor polynucleotide or of a target polynucleotide of an expression factor. Thus, RNAi of the invention may decrease or inhibit the expression of the expression factor and/or its target polynucleotide. The general method of making and using RNAi is disclosed in U.S. Pat. No. 5,506,559.

In one embodiment, RNAi containing a nucleotide sequence 100% identical to a portion of the target polynucleotide (for example, a gene encoding an efflux pump expression factor) may be used. In another embodiment, the RNAi has greater than 90% sequence identity with a portion of the target polynucleotide. In a further embodiment, RNAi with greater than 80%, 70%, 60% or 50% sequence identity with a portion of the target polynucleotide may be used. Sequence identity may be optimized by sequence comparison and alignment algorithms known in the art and calculating the percent difference between the nucleotide sequences by, for example, the Smith-Waterman algorithm as implemented in the BESTFIT software program using default parameters (e.g., University of Wisconsin Genetic Computing Group). Other methods known in the art may be used. The length of the identical nucleotide sequences may be at least 25, 50, 100, 200, 300 or 400 bases.

The RNAi may comprise one or more strands of polymerized ribonucleotide. RNAi may include modifications to either the phosphate-sugar backbone or the nucleoside. The double-stranded structure may be formed by a single self-complementary RNA strand or two complementary RNA strands. RNA duplex formation may be initiated either inside or outside the cell. The RNA strands need not be polyadenylated and the RNA strands need not be capable of being translated into a polypeptide by a cell's translational apparatus. The RNA may be synthesized either in vivo or in vitro as described above for antisense oligonucleotides. Alternatively, RNA may be chemically or enzymatically synthesized by manual or automated reactions.

Expression factor inhibitors of the invention also encompass chemicals, naturally-occurring, recombinant, or synthetic peptides, polypeptides, proteins, polysaccharides, small molecules and other compounds designed to reduce or inhibit expression factor activity or expression in a cell. These expression factor inhibitors may, for example, reduce or inhibit activity of an expression factor by interfering with a pathway that leads to activation or expression of the expression factor. The expression factor inhibitors of the invention may also reduce or inhibit the expression or activity of a polynucleotide or protein modulated by the expression factor by directly binding to the expression factor. For example, the expression factor inhibitor may be designed to bind to the MSE site on the gene of an efflux pump or efflux pump expression factor. The efflux pump expression factor inhibitor may mimic the efflux pump expression factor so that it binds to the MSE site on the efflux pump gene but does not activate its expression. In another embodiment, the expression factor inhibitor may act by preventing phosphorylation or dephosphorylation of the expression factor.

Peptides, polypeptides, and proteins that inhibit the activity or expression of an expression factor may be generated according to methods known in the art. For example, phage peptide display libraries can be used to express large numbers of peptides that can be screened in vitro to identify peptides that specifically bind the expression factor or inhibit the activity of the expression factor. Phage display technology provides a means for expressing a diverse population of random or selectively randomized peptides. Various methods of phage display and methods for producing diverse populations of peptides are well known in the art. For example, Ladner et al. (U.S. Pat. No. 5,223,409), describes methods for preparing diverse populations of binding domains on the surface of a phage. Ladner et al. describe phage vectors useful for producing a phage display library, as well as methods for selecting potential binding domains and producing randomly or selectively mutated binding domains. Screening of a phage display library generally involves in vitro panning of the library using a purified target molecule. Phage that bind the target molecule can be recovered, individual phage can be cloned and the peptide expressed by a cloned phage can be determined.

Similarly, Smith and Scott, Meth. Enzymol. 217:228-257, 1993, and Science 249:386-390, 1990, describe methods of producing phage peptide display libraries, including vectors and methods of diversifying the population of peptides that are expressed. See also, WO 91/07141 and WO 91/07149. Phage display technology can be particularly powerful when used, for example, with a codon based mutagenesis method, which can be used to produce random peptides or randomly or desirably biased peptides. See, e.g., U.S. Pat. No. 5,264,563. These and other well known methods can be used to produce a phage display library, which can be subjected to an in vitro panning method in order to identify a peptide, polypeptide, or protein that binds to an expression factor.

Peptides, polypeptides, proteins, polysaccharides, and the like that bind an expression factor or inhibit the activity of an expression factor may also be isolated from natural sources, and then optionally processed (e.g., via peptide cleavage) or, alternatively, synthesized by conventional techniques known in the art such as solid phase synthesis or recombinant expression. See, e.g., See Sambrook et al., Molecular Cloning: A Laboratory Manual, Vols 1-3 (2d ed. 1989), Cold Spring Harbor Laboratory Press. Automatic peptide synthesis can be performed using commercially available apparatus from manufacturers such as Applied Biosystems (Foster City, Calif.), and methods of doing so are well established. Recombinant production of the proteins may be in prokaryotic, such as phage or bacterial cells or eukaryotic systems, such as yeast, insect, or mammalian cells. Alternatively, proteins can be produced using cell-free in vitro systems known in the art.

A peptide, polypeptide, or protein that binds to an expression factor or inhibits the activity of an expression factor may be expressed as a fusion protein with a heterologous peptide. The peptide, polypeptide, or protein of the invention may be linked at its amino terminus, its carboxyl terminus, or both to a heterologous peptide. Optionally, multiple repeats of the heterologous peptide can be present in the fusion protein. Optionally, a peptide, polypeptide, or protein of the invention may be linked to multiple copies of a heterologous peptide, for example, at both the N and C termini of the heterologous peptide. Some heterologous proteins serve to enhance the half-life of the fused peptide, polypeptide, or protein, thereby increasing therapeutic efficacy in vivo. See, e.g., U.S. Pat. Nos. 5,876,969 and 5,565,335.

Other expression factor inhibitors may be identified by screening for chemicals and naturally-occurring, recombinant, or synthetic peptides, polypeptides, proteins, or polysaccharides for their ability to inhibit the expression or activity of an expression factor.

Expression factors may be screened for modulation of the expression of a target polynucleotide to which the expression factor is expected to bind, by introducing a reporter gene under the control of the promoter of the target polynucleotide into a host cell, introducing a candidate expression factor polynucleotide into the host cell, and detecting the expression of the reporter gene. In an embodiment of the invention, the host cell is a fungal host cell. In another embodiment, the target polynucleotide is CDR1 and therefore, the promoter is the CDR1 efflux pump promoter.

A “reporter gene” includes any polynucleotide which encodes an easily detectable product, including drug resistance, which polynucleotide is operably linked to a promoter. Operably linked refers to appropriate conditions in which an RNA polymerase may bind to the promoter of the regulatory region and proceed to transcribe the nucleotide sequence of the reporter gene. Commonly used reporter genes include polynucleotides that encode β-galactosidase, green fluorescent protein (GFP), and neomycin-resistance. Methods of detecting these products is well known in the art. The method of the invention allows for the screening of repressors and activators of gene expression.

An expression vector may be constructed to comprise any one or more of a nucleic acid encoding a reporter gene, an expression factor, or an expression factor inhibitor. An appropriately constructed expression vector may contain, for example, an origin of replication for autonomous replication in the host cells, one or more selectable markers, a limited number of useful restriction enzyme sites, a potential for high copy number, and one or more active promoters. Expression vectors may originate from a variety of sources such as viruses, plasmids, or the cells of a higher organism, such as yeast and mammalian cells.

The vectors of the invention may be introduced in vitro for in vitro testing for expression factor activity. Expression factor inhibitors, such as antisense oligonucleotides, RNAi, and polynucleotides coding for a peptide, protein, or polypeptide, may also be introduced into host cells via expression vectors. Methods of introducing the vectors include viral-based approaches and nonviral approaches, such as lipofection, ligand-DNA conjugates and direct injection of naked DNA. See, e.g., U.S. Pat. No. 6,140,484. Other methods described herein or known in the art may be used.

Polynucleotides may also be screened for the presence of one or more mid-sporulation elements (MSE). MSEs have been found in the S. cerevisiae NDT80 and also in the C. albicans homolog, CaNDT80. Thus, MSEs may play an important role in drug susceptibility and/or virulence of cells and identification of other polynucleotides containing MSEs may provide additional targets for various treatments. The polynucleotides may be screened using genomic or cDNA libraries obtained from the cells. Methods of making and obtaining genomic or cDNA libraries are know in the art. Bioinformatics has greatly improved the efficiency by which large number of polynucleotides, proteins, and small molecules may be screened. DNA microarrays may be used to screen for polynucleotides containing MSEs. Microarrays may also be used to screen for proteins that bind to polynucleotides containing MSEs. Microarrays are reviewed in, for example, Taton et al., Science 289:1757-1760, 2000; Reichert et al., Anal. Chem. 72:6025-6029, 2000; Lockhart et al., Nature 405:827-836, 2000; The Chipping Forecast, Nature Genetics 2:Supp., January 1999. Expression factor inhibitors may then be designed to interfere with the functioning of those polynucleotides containing MSEs.

The present invention also relates to the treatment of a patient, or for the benefit of a patient, by administration of an effective amount of at least one expression factor inhibitor. An effective amount of at least one drug may be also be administered and may be administered simultaneously or sequentially with the expression factor inhibitor. An “effective amount” refers to an amount sufficient to inhibit the expression of a polynucleotide in a patient. An “effective amount” also refers to an amount sufficient to reduce polynucleotide or protein activity, to ameliorate symptoms in a patient, to reduce or eliminate microbial infection in a patient, or to inhibit cell growth.

The present invention also provides compositions useful in carrying out the methods of the invention. The composition may comprise at least one expression factor inhibitor. In another embodiment, the composition may comprise at least one expression factor inhibitor and at least one drug. The drug may be an antimicrobial or antifungal agent. The compositions of the invention may also be used in appropriate association with other pharmaceutically active compounds. The following methods and excipients are merely exemplary and are in no way meant to be limiting.

The oral compositions may be used alone or in combination with appropriate additives to make tablets, powders, granules, or capsules, for example, with conventional additives, such as lactose, mannitol, corn starch, or potato starch; with binders, such as crystalline cellulose, cellulose derivatives, acacia, corn starch, or gelatins; with disintegrators, such as corn starch, potato starch, or sodium carboxymethylcellulose; with lubricants, such as talc or magnesium stearate; and if desired, with diluents, buffering agents, moistening agents, preservatives, and flavoring agents.

The compositions may include, depending on the composition desired, physiologically acceptable, nontoxic carriers, which are defined as vehicles commonly used to formulate compositions for animal or human administration. In general, the carrier is also pharmacologically acceptable, i.e., it does not affect the biological activity of the combination. Examples of such carriers are distilled water, physiological phosphate-buffered saline, Ringer's solution, dextrose solution, and Hank's balanced salt solution. Also included may be carrier molecules such as proteoglycans. Specific examples of such carrier molecules include, but are not limited to, glycosaminoglycans such as heparin sulfate, hyaluronic acid, keratin-sulfate, chondroitin 4-sulfate, chondroitin 6-sulfate, heparin sulfate and dermatin sulfate, perlecan, and pento polysulfate. In addition, the composition may include other excipients, adjuvants, or nontoxic, nontherapeutic, nonimmunogenic stabilizers, and the like.

The composition of the invention may be formulated into preparations for injection by dissolving, suspending, or emulsifying the composition in a physiologically acceptable carrier. Carriers include sterile liquids, such as water, oils, with or without the addition of a surfactant, and glycols. Oils may be petroleum derivatives, or of animal, vegetable, or synthetic origin, for example, peanut oil, soybean oil, and mineral oil. Examples of glycols include propylene glycol and polyethylene glycol. The compositions may also contain conventional additives such as solubilizers, isotonic agents, suspending agents, emulsifying agents, stabilizers, and preservatives. The composition of this invention may also be used in a sustained release form, for example, a depot injection, implant preparation, or osmotic pump, which can be formulated in such a manner as to permit a sustained release of the active ingredients.

The composition of the invention may be utilized in an aerosol composition to be administered via inhalation or pulmonary delivery. The composition of the present invention may be formulated into pressurized propellants such as dichlorodifluoromethane, nitrogen, and the like.

Administration of the composition of the invention may be accomplished by any convenient means, including parenteral injection, and may be systemic or localized in delivery. Administration of the composition may be achieved in various ways, including oral, buccal, rectal, parenteral, intraperitoneal, intradermal, transdermal, intratracheal, intrathecal, intranasal, gastric, intramuscular, intracranial, subdermal, etc., administration. The active agent(s) may be systemic after administration or may be localized by the use of regional administration, intramural administration, or use of an implant that acts to retain the active components at the site of implantation.

Unit dosage forms for oral or rectal administration such as syrups, elixirs, and suspensions may be provided wherein each dosage unit, for example, teaspoonful, tablespoonful, tablet, or suppository, contains a predetermined amount of the composition. Similarly, unit dosage forms for injection or intravenous administration may comprise the composition of the present invention as a solution in sterile water, normal saline, or another pharmaceutically acceptable carrier.

Those of skill in the art will readily appreciate that dose levels can vary as a function of the specific composition, the severity of the symptoms, and the susceptibility of the subject to side effects. Additionally, some of the specific compositions of the invention may be more potent than others. Dosages for a given composition are readily determinable by those of skill in the art by a variety of means, for example by measuring the relative physiological potency of a given composition by methods known in the art with respect to the potency of another composition and adjusting the dosage accordingly.

Other than in the operating examples, or where otherwise indicated, all numbers expressing quantities of ingredients, reaction conditions, and so forth used in this application are to be understood as being modified in all instances by the term “about.” Accordingly, unless the contrary is indicated, the numerical parameters set forth in this application are approximations that may vary depending upon the desired properties sought to be obtained by the present invention. At the very least, and not as an attempt to limit the application of the doctrine of equivalents to the scope of the claims, each numerical parameter should be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements.

Unless defined otherwise, all technical and scientific terms used herein have the same meaning as commonly understood by one of ordinary skill in the art to which this invention belongs. Although any methods and materials similar or equivalent to those described herein can also be used in the practice of the present invention, exemplary methods and materials are described for illustrative purposes.

All publications mentioned in this application are incorporated by reference to disclose and describe the methods and/or materials in connection with which the publications are cited. Additionally, the publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It must be noted that as used herein and in the appended claims, the singular forms “a”, “and”, and “the” include plural referents unless the context clearly dictates otherwise. Thus, for example, reference to “an antimicrobial agent” includes a plurality of antimicrobial agents and reference to “a fungal cell” includes reference to one or more fungal cells and equivalents thereof known to those skilled in the art.

Methods, techniques, and/or protocols (collectively “methods”) that can be used in the practice of the invention are not limited to the particular examples of these procedures cited throughout the specification but embrace any procedure known in the art for the same purpose. Furthermore, although some methods may be described in a particular context in the specification, their use in the instant invention is not limited to that context.

The present invention is illustrated by the following Examples, which are not intended to be limiting in any way.

### EXAMPLE 1

### Construction of a Leu2+ CDR1p-lacZ Expression Vector

An expression vector in which the lacZ gene (expressing β-galactosidase, or β-gal) (Myers et al., Gene 45:299-310, 1986) was used as the reporter gene and placed under the control of the CDR1 promoter (CDR1p) was constructed in order to monitor the expression from the CDR1 promoter.

A 1.2 kb DNA fragment containing the promoter and the translation initiation codon ATG of CDR1 was amplified from the genome of the C. albicans Sc5314 laboratory strain by polymerase chain reaction (PCR). The PCR fragment was generated using the primers HJL21, 5′ d(TTTCCCGGGGGAT CCTCGTTACTCAA) and HJL22, 5′ d(CCCAAGCTTGCATAATTTTTTTCTTTTT GACCT), which introduced a 5′ XmaI and a 3′ HindIII site (underlined), respectively. The PCR fragment was inserted into the Xma I and Hind III cloning sites of plasmid YIp363 (Myers et al., Gene 45:299-310, 1986) to place the CDR1 promoter fragment in-frame with the lacZ gene. The resultant plasmid was designated LOB42.

The primers HJL44, 5′ d(TTTTCCCGGGCAGCAGTTTAGAAGCAAT) and HJL45, 5′ d(CCCCCCCGGGTGATTTGTCTTAACATT) were used to amplify a DNA fragment containing the ADE3 gene from 37 to 1648 bp. This DNA fragment of the ADE3 gene was cloned into the XmaI of the LOB42 plasmid in an antisense orientation to the CDR1p-lacZ fusion to create the LOB43 plasmid. The LOB43 construct was digested with XhoI to linearize the DNA at the 337 bp downstream of the translation initiation site of the ADE3 gene and transformed into S. cerevisiae strain 10560-2B (FIG. 4). The CDR1p-lacZ fusion of the LOB43 plasmid was integrated into the ADE3 locus through homologous recombination to produce the Leu2⁺ transformant, the SLO1 strain. The wild type LEU2 gene on the YIp363 plasmid was used as the selective marker.

### EXAMPLE 2

### Construction and Screening of a C. albicans Genomic Library

A C. albicans genomic library was constructed in a high-copy number S. cerevisiae vector, 2μ-URA3 (Liu et al., Science 266:1723-1726, 1994). The library was transformed into strain SLO1 described in Example 1 by standard techniques. A control strain, SLO2, was created by transforming SLO1 with the control vector, 2μ-URA3. The lacZ gene was used as a reporter to monitor the activity of the CDR1 promoter and its β-galactosidase (β-Gal) activity was determined using the filter assay and/or liquid assay described previously (Lundblad, Current Protocols in Molecular Biology, 2 , pp. 3.6.2-3.6.5. John Wiley & Sons, New York, N.Y., 1997; Mosch et al., Proc. Natl. Acad. Sci. U.S.A. 93:5352-5356, 1996).

Library transformants (approximately 500 colonies per 150 mm plate) were grown for three days before being replica-plated onto filter members laid on top of agar medium. URA+ colonies with elevated β-Gal activity compared to the control strain with only 2μ of plasmid in the filter assay were selected for further screening. If β-Gal activity of the colony returned to basal level upon removal of the library plasmid from the cells, it was considered a clone harboring a candidate plasmid for a trans-regulatory factor of CDR1. In order to remove the library plasmid from the candidate colonies, the colonies were separately grown on YPD liquid medium for two days. The β-Gal activities of the candidate colonies and those which have lost the library plasmids were compared in the β-Gal liquid assay.

Using the above method, approximately 24,000 independent library transformant colonies covering about three times the C. albicans genome size were generated. Of the 74 candidates picked initially, sixteen had β-Gal activity in the second filter assay. Among them, five had higher β-Gal activity than the others and were chosen for further analysis. Two of these candidates contained mutations in the S. cerevisiae chromosome because the strains still had the same level of β-Gal activity after they were induced to lose the library plasmid. In contrast, the β-Gal activity of the remaining three candidates were reduced to basal level when they were induced to lose their library plasmids. Two out of the three candidates harbored the same plasmid, which was designated LOB44 . The URA+ colony harboring LOB44 was designated SLO3.

Analysis of the LOB44 plasmid revealed that it contained an insert of approximately 5 kb, which comprised two full-length open reading frames (ORFs), one for CaNdt80p and the other for orf6.1265, a short hypothetical protein with 106 amino acids in length. Plasmid LOB45 was generated to compare the activity from the 2.8 kb genomic fragment of the CaNDT80 gene from C. albicans strain SC5314 to that of plasmid LOB44. To create plasmid LOB45, primers HJL72, 5′ d(CGGGATCCTTGTGGCGATTTTCACTTTC) and HJL73, 5′ d(CCGGATCCTCAATGGGGGTGGATTGA) were used to amplify the CaNDT80 gene of C. albicans from strain SC5314. The primers created convenient BamHI sites for cloning (underlined). The amplified DNA fragment begins from 578 bp upstream of the predicted start codon (ATG) of the CaNDT80 gene to 479 bp downstream of the predicted stop codon (TAA) of CaNDT80. The amplified DNA fragment was digested with BamHI and introduced into the BamHI site of the pRS426 vector (Christianson et al., Gene 110:119-122, 1992) to generate the LOB45 plasmid. LOB45 was transformed into the SLO1 strain described above, and the transformant was designated SLO5.

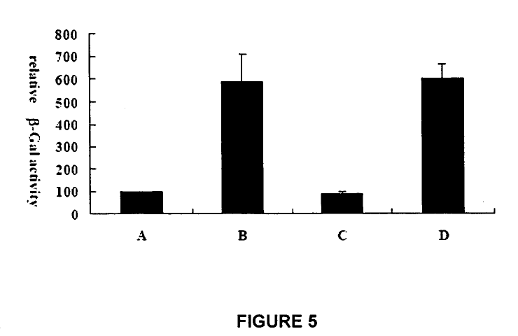

SLO3 was tested in a quantitative liquid assay (FIG. 5). SLO3 (lane B) showed about six-fold greater β-gal activity compared with the control, SLO2 (lane A). Removal of the LOB44 plasmid from the SLO3 strain (land C) reduced the β-gal activity to basal level (lane A). Re-introduction of the LOB44 plasmid into the parental SLO1 strain restored β-gal activity to the previous level (lane D).

Additionally, the β-gal activity of SLO5 strain harboring plasmid LOB45 was as high as that of SLO3. These results demonstrate that increase in β-gal activity from the CDR1p-lacZ insert in S. cerevisiae is dependent on CaNDT80.

### EXAMPLE 3

### Overexpression of CaNDT80 Protein (CaNdt80p) Reduces Drug Susceptibility

The effect on drug susceptibility by overexpression of CaNdt80p and its S. cerevisiae homolog, Ndt80p, were compared using the antifungal strips, Etest (AB BIODISK, Solna, Sweden) (Arendrup et al., J. Antimicrob. Chemother. 47:521-526, 2001; Chang et al., J. Clin. Microbiol 39:1328-1333, 2001). S. cerevisiae Ndt80p was identified as a meiosis specific transcription factor (Chu et al., Science 282:699-705, 1998; Chu and Herskowitz, Mol. Cell 1:685-696, 1998). Ndt80p is autoregulated and activates its targets through a mid-sporulation element consensus site (MSE: gNCRCAAAA/T) in the target (Chu and Herskowitz, Mol. Cell 1:685-696, 1998). Expression of the NDT80 gene is repressed during vegetative growth (Xu et al., Mol. Cell Biol. 15:6572-6581, 1995; Pak and Segall, Mol. Cell Biol. 22:6417-6429, 2002).

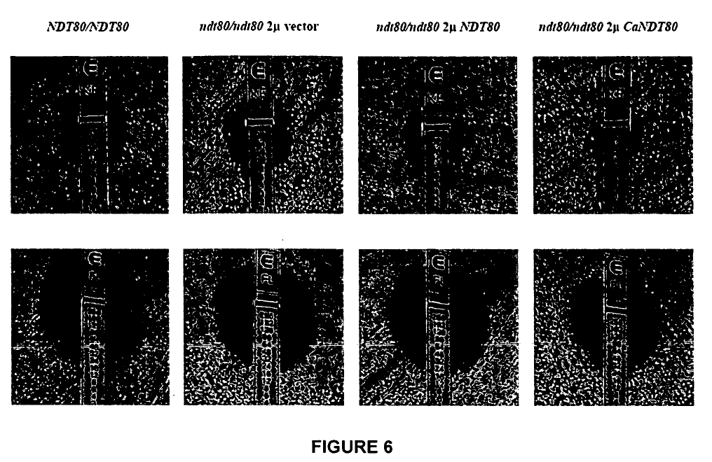

First, homogenized isolated colonies from an overnight SD plate were transferred into a 0.85% NaCl solution to achieve a suspension containing 5×10⁶ cells/ml. A sterile swab was dipped into the inoculum suspension and the entire agar surface of an SD plate was streaked evenly with the swab. The plates were allowed to dry until all excess moisture was completely absorbed, and the Etest strips (AB BIODISK, Solna, Sweden) were applied onto each plate. The plates were incubated at 30° C. and were photographed after 3 days of growth. These Etest strips tested the susceptibility to ketoconazole (0.002-32 μg/ml) (KE) and fluconazole (0.016-256 μg/ml) (FL). Susceptibility is indicated by a clear ring around the antifungal strips where growth has been inhibited. Degree of susceptibility is indicated by the size of the ring. The strains tested were the NDT80/NDT80 wild type S. cerevisiae strain transformed with the control vectors 2μ-URA3and CEN-LEU2 (Christianson et al., Gene 110:119-122, 1992; Sikorski and Hieter, Genetics 122:19-27, 1989) (designated as strain SLO16) (“NDT80/NDT80” lane in FIG. 6); a ndt80/ndt80 homozygous mutant transformed with the control 2μ-URA3 vector (designated as strain SLO14) (“ndt80/ndt80 2μ vector” lane in FIG. 6); a ndt80/ndt80 homozygous mutant transformed with the 2μ-URA3-NDT80 plasmid, LOB46 (designated as strain SLO13) (“ndt80/ndt80 2μ NDT80” lane in FIG. 6); and a ndt80/ndt80 homozygous mutant transformed with the 2μ-URA3-CaNDT80 plasmid, LOB44 (designated as strain SLO15) (“ndt80/ndt80 2μ CaNDT80” lane in FIG. 6).

The homozygous ndt80/ndt80 mutant S. cerevisiae strain was created by amplifying by PCR a LEU2 DNA fragment from plasmid pRS305 (Sikorski and Hieter, Genetics 122:19-27, 1989), and flanked by a 65 bp 5′ untranslated region (UTR) and 3′ UTR of the NDT80 polynucleotide. This DNA fragment was transformed into S. cerevisiae strain 10560-2B (MATa his3::hisG URA3-52 leu2::hisG) to generate a ndt80::LEU2 strain, SLO6 (MATa ndt80::LEU2 his3::hisG URA3-52 leu2::hisG). SLO6 was mated with strain 10560-5B (MAT α trp1::hisG URA3-52 leu2::hisG) to yield SLO7 (MATa ndt80::LEU2 his 1::hisG URA3-52 leu2::hisG), SLO8 (MAT α ndt80::LEU2 trp1::hisG URA3-52 leu2::hisG), SLO9 (MATa trp1::hisG URA3-52 leu2::hisG) and SLO10 (MAT α, his1::hisG, URA3-52, leu2::hisG). SLO7 and SLO8 were mated to yield diploid strain SLO11 (MATa/MAT α ndt80::LEU2/ndt80::LEU2 URA3-52/URA3-52 leu2::hisG/leu2::hisG). SLO9 and SLO10 were mated to yield diploid strain SLO12 (MATa/MAT α URA3-52/URA3-52 leu2::hisG/leu2::hisG). A 3.6 kb PCR fragment containing the NDT80 gene was inserted into the vector 2μ-URA3 (pRS426) (Christianson et al., Gene 110:119-122, 1992), to generate the 2μ-URA3-NDT80plasmid, LOB46. Strains SLO13, SLO14 and SLO15 were created by transforming the vectors 2μ-URA3 (pRS426), 2μ-URA3-NDT80 plasmid (LOB46), and 2μ-URA3-CaNDT80 plasmid (LOB44), respectively into SLO11. Strain SLO16 is a strain in which SLO12 was transformed with both 2μ-URA3 (pRS426) and 2μ-HIS3 (pRS315) (Sikorski and Hieter, Genetics 122:19-27, 1989).

As FIG. 6 shows, neither overexpression of then S. cerevisiae NDT80 gene (“ndt80/ndt80 2μ NDT80”) nor mutations on the NDT80 gene (“ndt80/ndt80 2μ vector”) altered drug susceptibility, which is consistent with the fact that Ndt80p is active in S. cerevisiae diploid cells only during meiosis. By contrast, overexpression of the CaNDT80 gene decreased ketoconazole susceptibility in S. cerevisiae (compare “NDT80/NDT80” and “ndt80/ndt80 2μ CaNDT80”).

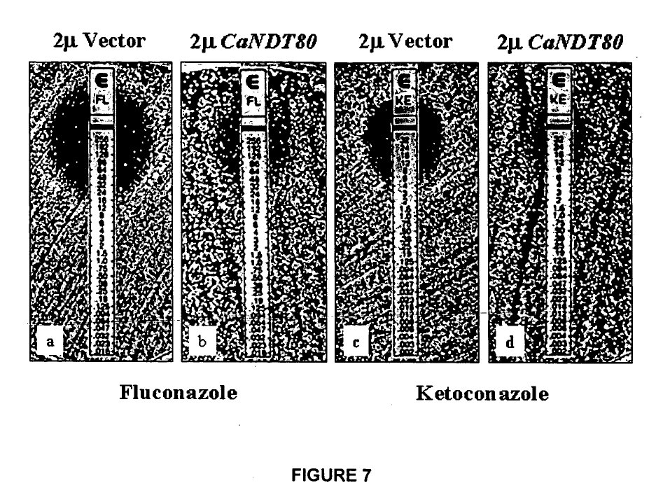

In a separate experiment, a strain harboring the LOB44 plasmid without the NDT80 gene was tested in the Etest. FIG. 7 shows that overexpression of CaNDT80 decreased the susceptibility to both fluconazole and ketoconazole in S. cerevisiae, compared to a strain transformed with a control vector. The minimum inhibitory concentration (MIC) of fluconazole increased from 24 μg/ml to 64 μg/ml when cells overexpressed CaNDT80 (FIG. 7B). Similarly, the MIC of ketoconazole increased from 6 μg/ml to equal or larger than 32 μg/ml when cells overexpressed CaNDT80 (FIG. 7D). The strain containing the LOB45 plasmid had a similar susceptibility to antifungal drugs as the LOB44 plasmid. These data suggest that CaNdt80p of C. albicans is capable of activating the gene involved in drug resistance in S. cerevisiae.

### EXAMPLE 4

### Inactivation of CaNDT80 Increases Drug Susceptibility

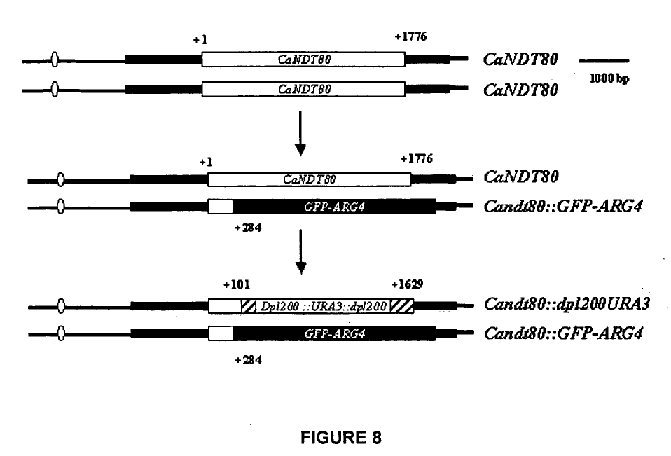

A homozygous C. albicans mutant Candt80/Candt80 was generated and tested for susceptibility to antifungal drugs. The homozygous Candt80/Candt80 mutant was constructed based on the gene disruption method described in the art (Wilson et al., Yeast 16: 65-70, 2000; Gerami-Nejad et al., Yeast 18:859-864, 2001; Wilson et al., J Bacteriol. 181:1868-1874, 1999) and as shown in FIG. 8. Plasmids pGFP-ARG4 and pDDB57 containing the URA3-dpl200based cassette (Wilson et al., Yeast 16: 65-70, 2000) were used as PCR templates to construct the heterozygous and homozygous mutants of CaNDT80, respectively. The PCR product contained the GFP-ARG4 fragment flanked by short CaNDT80 homology regions of 70 bp at each end and was transformed into strain BWP17 (URA3Δ::λimm434/URA3Δ::λimm434 his1::hisG/his1::hisG arg4::hisG/arg4::hisG) to knockout the CaNDT80 gene, from 283 bp downstream of the translation initiating codon ATG to 359 bp downstream of the stop codon. Transformants were Arg+ and designated as strain YLO131 (CaNDT80/Candt80::GFP-ARG4), a heterozygous mutant for CaNDT80. The same PCR product was transformed into YLO131 to knockout the second copy of the CaNDT80 gene, from 101 bp downstream of the translation initiating codon ATG to 148 bp upstream of the stop codon. The Candt80/Candt80 homozygous mutant strain was designated as YLO132.

Strain YLO0133 is a homozygous Candt80/Candt80 mutant strain in which the ENO1 promoter was disrupted. An AccI-digested pT7tetR-HIS1 (Nakayama et al., Infect. Immun. 68:6712-6719, 2000) was integrated into the ENO1 promoter of YLO132 to create the strain YLO133 (URA3Δ::λimm434/URA3Δ::λimm434 his1::hisG/his1::hisG arg4::hisG/arg4::hisG Candt80::GFP-ARG4/Candt80::URA3 eno1p::tetR-HIS1).

Strain YLO137 was generated to restore one copy of the CaNDT80 gene in the homozygous mutant strain, YLO132. A BamHI DNA fragment containing wild type CaNDT80 from plasmid LOB44 was inserted into the pGEM-HIS1 vector (Wilson et al., J. Bacteriol. 181:1868-1874, 1999) to generate pGEM-HIS1-CaNDT80, LOB49. The LOB49 plasmid was digested at the SpeI located in the promoter of CaNDT80 and was transformed into YLO132 to construct strain YLO137 (URA3Δ::λimm434/URA3Δ::λimm434 his1::hisG/his1::hisG arg4::hisG/arg4::hisG Candt80::GFP-ARG4/Candt80::URA::CaNDT80::HIS1).

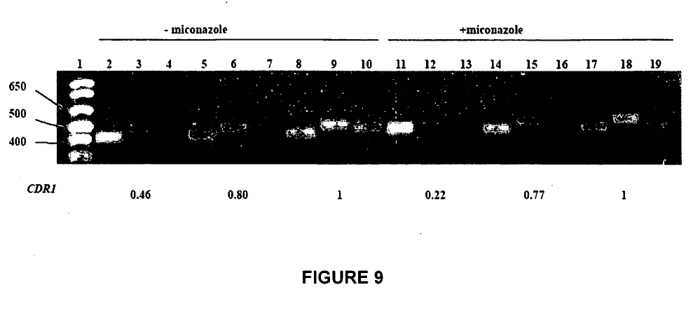

C. albicans strains SC5314 (wild type), YLO133 (homozygous mutant Candt80/Candt80) and YLO137 (rescued strain of YLO133 with one copy of CaNDT80), were grown in the presence or absence of the antifungal agent, miconazole (Sigma) at 100 μg/ml for one hour at 30° C. The level of mRNAs of TEF3 (control), CDR1, and CaNDT80, was measured by reverse transcriptase polymerase chain reaction (RT-PCR). FIG. 9 shows the level of CDR1 and CaNDT80 mRNAs in each of the strains that were normalized to those of the wild type. Lanes 2-4 and 11-13 represent mRNA from YLO133 (homozygous mutant), lanes 5-7 and 14-16 represent mRNA from YLO137 (rescued mutant), and lanes 8-10 and 17-19 represent mRNA from the wild-type strain, SC5314. Lanes 2, 5, 8, 11, 14, and 17 were tested for TEF3 mRNA (internal control); lanes 3, 6, 9, 12, 15, and 18, were tested for CDR1 mRNA; and lanes 4, 7, 10, 13, 16, and 19 were tested for CaNDT80 mRNA. As expected, no CaNDT80 mRNA was detected in the homozygous mutant (FIG. 9, lanes 3 and 13). In contrast, CaNDT80 mRNA was detected in the wild-type strain during vegetative growth (FIG. 9, lanes 10 and 19), unlike its homolog NDT80 in S. cerevisiae. The rescued mutant showed a lower level of CaNDT80 mRNA compared with wild type, but a greater level than in the homozygous mutant (FIG. 9, lanes 7 and 16). The patterns of mRNA isolated from different strains with or without miconazole treatment were comparable.

The level of CDR1 mRNA, which encodes the efflux pump, was also reduced in the homozygous mutant (FIG. 9, lanes 3 vs 9 and 12 vs 18). The result shows that overexpression of the CaNDT80 gene enhances the expression of the CDR1 efflux pump. However, the CaNDT80 protein may be only one of a number of factors that controls CDR1 expression because there was still a detectable level of CDR1 mRNA in the homozygous mutant strain (FIG. 9, lanes 3 and 12). Consistent results were obtained when mRNA levels were tested using real-time hot-start PCR performed with the LC FastStartDNA Master SYBR Green I kit in a LightCycler (Cat. No. 2239264, Roche, Germany).

The three C. albicans strains SC5314 (wild type), YLO133 (homozygous mutant Candt80/Candt80) and YLO137 (rescued strain of YLO133 with one copy of CaNDT80), were also tested for susceptibility to five different drugs using the Etest. Five different antifungal strips (AB BIODISK, Solna, Sweden) were used: amphotericin B at 0.002-32 μg/ml (AP), 5-fluorocytosine at 0.002-32 μg/ml (FC), fluconazole at 0.016-256 μg/ml (FL), itraconazole at 0.002-32 μg/ml (IT) and ketoconazole at 0.002-32 μg/ml (KE). Each strain was grown overnight on an SD plate and homogenized colonies from the plate were transferred into 0.85% NaCl to achieve a suspension of about 5×10⁶ cells/ml. A sterile swab was dipped into the inoculum suspension and the entire agar surface of a SD plate was evenly streaked. Each plate was dried until excess moisture was completely absorbed and each of the five different Etest strips were applied onto each plate. The plates were incubated at 30° C. and were photographed after 2 days of growth. Susceptibility to a drug is indicated by a clear inhibition zone around the antifungal strip where growth has been inhibited. Degree of susceptibility is indicated by the size of the ring.

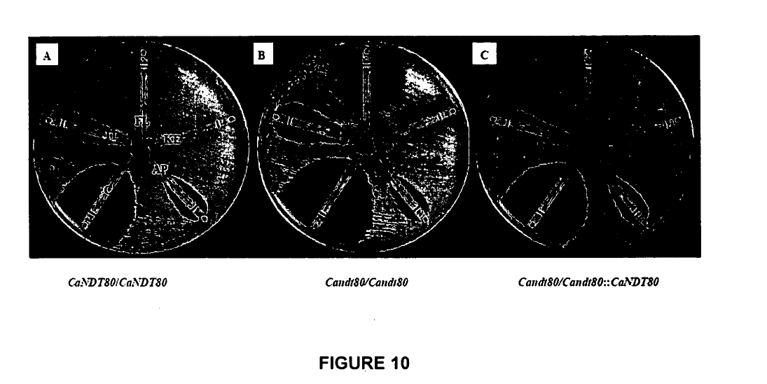

The homozygous mutant showed increased susceptibility to fluconazole, ketoconazole, and 5-fluorocytosine (compare FIGS. 10A and 10B). Moreover, the rescued strain YLO137, which has only one copy of CaNDT80, was more susceptible to fluconazole than the wild type strain, which has two copies of CaNDT80 (compare FIGS. 10A and 10C), suggesting a dosage effect of CaNDT80.

An agar dilution assay showed that mutations on either CDR1 or CaNDT80 made C. albicans more susceptible to the antifungal agents, fluconazole and voriconazole. The cdr1/cdr1 homozygous mutant was constructed according to Sanglard et al., Antimicrob. Agents Chemother. 40:2300-2305, 1996. Fluconazole and voriconazole were prepared to a final concentration of 25 μg/ml and 1 μg/ml, respectively in dimethylsulfoxide (DMSO). C. albicans cells grown on media containing DMSO and in the absence of drug, was used as the control. C. albicans cells were diluted to an OD₆₀₀ of 2 (corresponding to approximately 2×10⁷cells/ml) and 0.5 μl was spotted onto plates containing either fluconazole (25 μg/ml) or voriconazole (1 μg/ml) using a replica device (Oxoid Inc., Canada). Cells were also prepared and spotted in 10-fold serial dilutions (10⁴ to 10 cells).

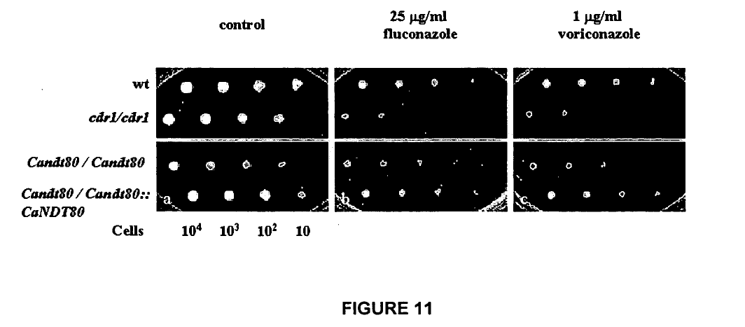

As FIG. 11 shows, cells grew in all spots in the absence of drugs (FIG. 11A). The Candt80/Candt80 mutant was more susceptible to both fluconazole and voriconazole than the wild-type (wt) or the rescued strain containing a copy of wild-type CaNDT80 (Candt80/Candt80::CaNDT80). Additionally, the cdr1/cdr1 mutant was more susceptible to the antifungal agents than the Candt80/Candt80 mutant. As FIGS. 11B and C show, less cdr1/cdr1 cells grew in the presence of the drugs at a cell concentration of 10³ and 10² than the Candt80/Candt80 cells.

### EXAMPLE 5

### The Candt80/Candt80 Homozygous Mutant is Avirulent

C. albicans and S. cerevisiae are capable of switching from a yeast to an infectious filamentous form, usually in the form of pseudohyphae or true hypae (Lo et al., Cell 90:939-949, 1997). The morphogenic switch in Saccharomyces is regulated by regulatory proteins, including Ste12p and Phd1p (Lo et al., Cell 90:939-949, 1997). Their Candida homologs, Cph1p and Efg1, have also been shown to regulate the morphogenic switch (Lo et al., Cell 90:939-949, 1997). Homozygous mutations at both loci produce Candida locked in the yeast form, and renders the Candida avirulent in a mouse model (Lo et al., Cell 90:939-949, 1997). The Candt80/Candt80 homozygous mutant was also tested for germ tube formation and virulence in the mouse model. Germ tube emergence occurs during the yeast-hypha transition and indicates an infectious form of the microbe.

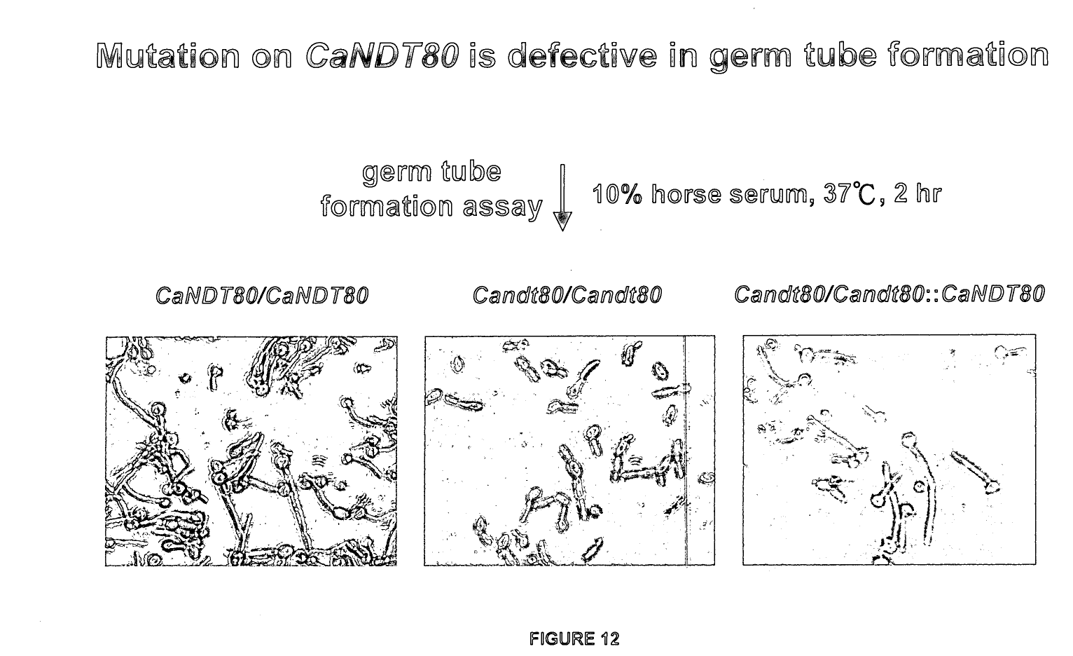

The Candt80/Candt80 homozygous Candida mutant was incubated in YPD liquid medium containing 10% horse serum for 2 hours at 37° C. and viewed under a microscope. As shown in FIG. 12, the Candt80/Candt80 mutant failed to form germ tubes and hyphae under the conditions tested, while the CaNDT80/CaNDT80 wild type strain developed germ tubes and hyphae. The Candt80/Candt80 mutant could be rescued by complementation with a wild type CaNDT80 and developed germ tubes and hyphae. Thus, the CaNDT80 gene is involved in the morphogenic switch of Candida from yeast to filamentous form.

The Candt80/Candt80 mutant was also tested in a mouse model for Candida virulence as described in Lo et al., Cell 90:939-949, 1997. The Candida strains were tested for virulence by injecting 0.1 ml of cells (1×10⁶) into the tail vein of 4 BALB/c mice (Charles River Laboratories, Wilmington, Mass.) and survival was monitored daily.

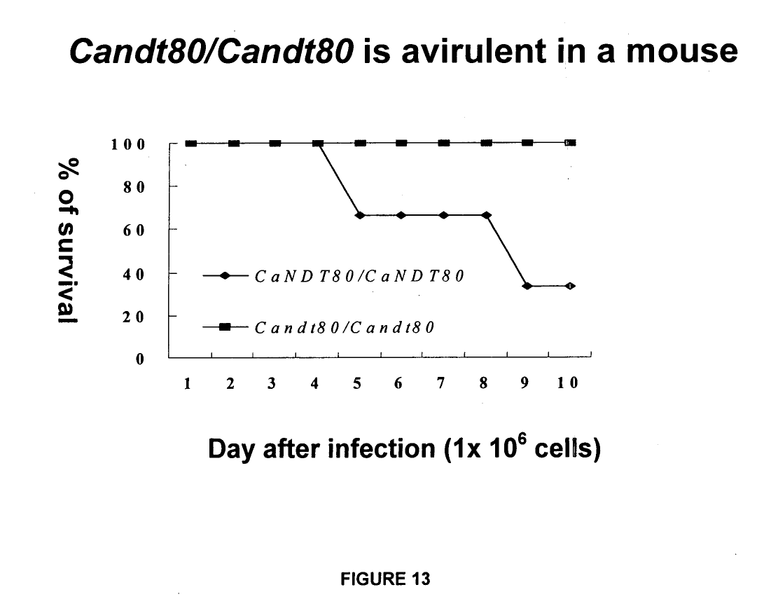

As FIG. 13 shows, wild-type Candida, CaNDT80/CaNDT80, was lethal: less than 50% of the mice survived 10 days after injection. In contrast, 100% of the mice injected with the homozygous Candt80/Candt80 mutant strain were surviving 10 days after injection.

As this Example illustrates, CaNDT80 is involved in the virulence of Candida and may be targeted for treatment of Candida infection.

### EXAMPLE 6

### Construction and Integration of MDR1p-lacZ Plasmids

Five different plasmids containing different lengths of the MDR1 promoter (approximately 300, 600, 900, 1200, and 2700 base pairs upstream of the translation initiation site of MDR1) were constructed. The different fragments of the MDR1 promoter was amplified by polymerase chain reaction (PCR) using different primer sets. In all reactions, the reverse primer, oligonucleotide HJL30 (5′-AACCCAAGCTTGCA TTGTGAAGTTCTATGT-3′), which contains a HindIII restriction enzyme site and which is complementary to the MDR1 promoter from +4 to −15 (where the A of the ATG translation initiation site is designated as +1), was used. Forward primers used for amplification of each promoter fragment were as follows:

LHL3 (complementary to −310 to −293):

5′-CGCGGATCCACCAATTAATCACAACGG-3′

LHL2 (complementary to −644 to −627):

5′-GCGGGATCCTCATGTAACCTTGCAATC-3′

LHL1 (complementary to −966 to −947):

5′-CGCGGATCCCTTAGACTTACTTATATCCG-3′

HJL31 (complementary to −1242 to −1224):

5′-CGCGGATCCGGCTTGCTAAACATTATCA-3′

HJL29 (complementary to −2696 to −2673):

5′-CGCGGATCCAGAGAATCCAGAAAAGAG-3′

The amplified promoter fragments were introduced in-frame into a lacZ fusion plasmid YEp363 and YIp363, which contains the wild type LEU2 gene as a selective marker (Myers et al., Gene 45:299-310, 1986). YEp363 is a plasmid that can replicate autonomously in E. coli and in yeast and is therefore useful for transient overexpression of a protein, whereas YIp363 can be used for integrating the plasmid sequences into a yeast host chromosome (Myers et al., Gene 45:299-310, 1986).

The MDR1p-lacZ plasmid containing the 2700 base-pair fragment of the MDR1 promoter was integrated into the ADE3 locus of S. cerevisiae through homologous recombination. First, a portion of the ADE3 gene was amplified by PCR using the following oligonucleotides:

HJL44 (complementary to positions +547 to +565 of the ADE3 gene):

5′-TTTCCGGGCAGCATTTAGAAGCAAT-3′

HLJ45 (complementary to positions +2143 to +2170 of the ADE3 gene):

5′-CCCCCCCGGGTGATTTGTCTTAACATT-3′

The 1.6 kb amplified DNA fragment of the ADE3 gene was cloned into the MDR1p-lacZ plasmid containing the 2700 base-pair fragment of the MDR1 promoter. The resulting integrative plasmid was transformed into S. cerevisiae and integrated into the yeast host chromosome at the ADE3 locus.

### EXAMPLE 7

### Screening for Trans-Regulatory Factors of the CDR1 Gene

The C. albicans genomic library prepared according to Example 2 was transformed into the S. cerevisiae strain containing the CDR1p-lacZ plasmid. Genes which activated gene expression of the lacZ gene were cloned. Two candidate genes, designated as REP1 (regulator of efflux pump 1) (see FIG. 3) and CaNDT80 (see FIG. 2), were identified by this procedure. REP1 increased β-galactosidase activity from the CDR1 promoter by six-fold when tested according to Example 2. Overexpression of REP1 also increased β-galactosidase activity from the MDR1 promoter by six-fold compared to the control. CaNDT80 increased β-galactosidase activity from the CDR1 promoter by six-fold compared to the control, but not from the MDR1 promoter. Overexpression of CaNDT80 was also found to decrease the susceptibility of S. cerevisiae to both fluconazole and ketoconazole, which is consistent with the observation that the CDR1 efflux pump transports fluconazole and ketoconazole.

The specification is most thoroughly understood in light of the teachings of the references cited within the specification, all of which are hereby incorporated by reference in their entirety. The embodiments within the specification provide an illustration of embodiments of the invention and should not be construed to limit the scope of the invention. The skilled artisan recognizes that many other embodiments are encompassed by the claimed invention and that it is intended that the specification and examples be considered as exemplary only, with the true scope and spirit of the invention being indicated by the following claims.

1. A method for inhibiting the growth of a cell, comprising contacting the cell with at least one efflux pump expression factor inhibitor, wherein the efflux pump expression factor inhibitor inhibits an efflux pump expression factor capable of binding to a midsporulation element (MSE) in a target polynucleotide.

2. The method of claim 1, wherein the efflux pump expression factor is CaNDT80, or a homolog or variant thereof, and modulates the expression of an efflux pump selected from CDR1, CDR2, and MDR1, or a homolog or a variant thereof.

3. The method of claim 1, wherein the cell is selected from a bacterial cell, fungal cell, yeast cell, and mammalian cell.

4. The method of claim 3, wherein the fungal cell is selected from a Candida species, Aspergillus species, and Cryptococcus species, or mixtures thereof.

5. The method of claim 4, wherein the Candida species is selected from C. albicans, C. krusei, C. tropicalis, and C. glabrata, or mixtures thereof.

6. The method of claim 3, wherein the mammalian cell is a cancer cell.

7. A method for inhibiting the growth of a cell, comprising contacting the cell with at least one efflux pump expression factor inhibitor, wherein the efflux pump expression factor inhibitor inhibits the expression or activity of REP1, or a homolog or variant thereof.

8. The method of claim 7, wherein REP1, or a homolog or variant thereof, modulates the expression of an efflux pump selected from CDR1, CDR2, and MDR1, or a homolog or a variant thereof.

9. A composition for inhibiting the growth of a cell, comprising at least one efflux pump expression factor inhibitor, wherein the efflux pump expression factor inhibitor inhibits an efflux pump expression factor capable of binding to a midsporulation element (MSE) in a target polynucleotide.

10. The composition of claim 9, wherein the efflux pump expression factor is CaNDT80, or a homolog or variant thereof, and modulates the expression of an efflux pump selected from CDR1, CDR2, and MDR1, or a homolog or a variant thereof.

11. The composition of claim 9, further comprising at least one drug.

12. The composition of claim 9, wherein the cell is selected from a bacterial cell, fungal cell, yeast cell, and mammalian cell.

13. The composition of claim 12, wherein the fungal cell is a Candida species, Aspergillus species, and Cryptococcus species, or mixtures thereof.

14. The composition of claim 14, wherein the Candida species is selected from C. albicans, C. krusei, C. tropicalis, and C. glabrata, or mixtures thereof.

15. The composition of claim 12, wherein the mammalian cell is a cancer cell.

16. A composition for inhibiting the growth of a cell, comprising at least one efflux pump expression factor inhibitor, wherein the efflux pump expression factor inhibitor inhibits the expression or activity of REP1, or a homolog or variant thereof.

17. The composition of claim 16, wherein REP1, or a homolog or variant thereof, modulates the expression of an efflux pump selected from CDR1, CDR2, and MDR1, or a homolog or a variant thereof.

18. A method for enhancing the activity of a drug in a cell, comprising:
a. inhibiting the expression or activity of an efflux pump expression factor capable of binding to a midsporulation element (MSE) in a target polynucleotide;
b. inhibiting the expression of at least one efflux pump polynucleotide in the cell;
c. contacting the cell with at least one drug; and
d. enhancing the activity of the drug.

19. The method of claim 18, wherein the efflux pump expression factor is CaNDT80, or a homolog or variant thereof, and wherein the efflux pump polynucleotide is selected from CDR1, CDR2, and MDR1, or a homolog or a variant thereof.

20. The method of claim 18, wherein the cell is selected from a bacterial cell, fungal cell, yeast cell, and mammalian cell.

21. The method of claim 20, wherein the fungal cell is selected from a Candida species, Aspergillus species, and Cryptococcus species, or mixtures thereof.

22. The method of claim 22, wherein the Candida species is selected from C. albicans, C. krusei, C. tropicalis, and C. glabrata, or mixtures thereof.

23. The method of claim 20, wherein the mammalian cell is a cancer cell.

24. The method of claim 18, wherein the drug is an antifungal agent and wherein the antifungal agent is an azole.

25. A method for enhancing the activity of a drug in a cell, comprising:
a. inhibiting the expression or activity of REP1, or a homolog or variant thereof;
b. inhibiting the expression of at least one efflux pump polynucleotide in the cell;
c. contacting the cell with at least one drug; and
d. enhancing the activity of the drug.

26. The method of claim 25, wherein the efflux pump polynucleotide is selected from CDR1, CDR2, and MDR1, or a homolog or variant thereof.

27. A method for screening for a polynucleotide encoding an efflux pump expression factor in a cell, comprising:
a. introducing a reporter gene under the control of an efflux pump promoter into a host cell;
b. introducing a candidate efflux pump expression factor polynucleotide into the host cell of step (a); and
c. detecting the expression of the reporter gene.

28. The method of claim 27, wherein the efflux pump is selected from CDR1, CDR2, and MDR1, or a homolog or variant thereof.

29. The method of claim 27, wherein the host cell is selected from a bacterial cell, fungal cell, yeast cell, and mammalian cell.

30. The method of claim 29, wherein the yeast host cell is a Saccharomyces species.

31. A method of treating a patient with a disease, disorder, or infection, comprising: administering to the patient an effective amount of at least one expression factor inhibitor, wherein the efflux pump expression factor inhibitor inhibits an efflux pump expression factor capable of binding to a midsporulation element (MSE) in a target polynucleotide.

32. The method of claim 31, further comprising administering to the patient at least one drug.

33. The method of claim 31, wherein the infection is selected from a bacterial infection, fungal infection, and yeast infection.

34. The method of claim 33, wherein the fungal infection is a C. albicans infection.

35. The method of claim 31, further wherein the efflux pump expression factor is CaNDT80, or a homolog or variant thereof, and modulates the expression of an efflux pump selected from CDR1, CDR2, and MDR1, or a homolog or a variant thereof.

36. A method of treating a patient with a disease, disorder, or infection, comprising: administering to the patient an effective amount of at least one expression factor inhibitor, wherein the expression factor inhibitor inhibits the expression or activity of REP1, or homolog or variant thereof.

37. The method of claim 36, wherein the expression of an efflux pump selected from CDR1, CDR2, or MDR1, or a homolog or variant thereof, is inhibited.

38. A pharmaceutical composition comprising at least one efflux pump expression factor inhibitor, wherein the efflux pump expression factor inhibitor inhibits an efflux pump expression factor capable of binding to a midsporulation element (MSE) in a target polynucleotide.

39. The pharmaceutical composition of claim 38, wherein the efflux pump expression factor is CaNDT80, or a homolog or variant thereof, and modulates the expression of an efflux pump selected from CDR1, CDR2, and MDR1, or a homolog or a variant thereof.

40. The pharmaceutical composition of claim 38, further comprising at least one drug.

41. The pharmaceutical composition of claim 38, 39, or 40, further comprising a pharmaceutically acceptable carrier.

42. A pharmaceutical composition comprising at least one efflux pump expression factor inhibitor, wherein the efflux pump expression factor inhibitor is an inhibitor of expression or activity of REP1, or a homolog or variant thereof.

43. The pharmaceutical composition of claim 42, further comprising a pharmaceutically acceptable carrier.

44. A method for inhibiting the growth of a cell, comprising:
a. inhibiting the binding of an expression factor to a mid-sporulation element (MSE); and
b. inhibiting the growth of a cell.

45. The method of claim 44, wherein the cell is selected from a bacterial cell, fungal cell, yeast cell, and mammalian cell.

46. The method of claim 45, wherein the fungal cell is selected from a Candida species, Aspergillus species, and Cryptococcus species, or mixtures thereof.

47. The method of claim 46, wherein the Candida species is selected from C. albicans, C. krusei, C. tropicalis, and C. glabrata, or mixtures thereof.

48. The method of claim 45, wherein the mammalian cell is a cancer cell.

49. The method of claim 44, wherein the expression factor is CaNDT80, or a homolog or variant thereof.

50. The method of claim 44, wherein the MSE is from a polynucleotide selected from CaNDT80, CDR1, CDR2, and MDR1, or a homolog or variant thereof.

51. The method of claim 44, further comprising contacting the cell with at least one drug.

52. A method for inhibiting virulence of a cell, comprising:
a. contacting the cell with at least one efflux pump expression factor inhibitor; and
b. inhibiting virulence of a cell;
wherein the efflux pump expression factor inhibitor inhibits an efflux pump expression factor capable of binding to a midsporulation element (MSE) in a target polynucleotide.

53. The method of claim 52, wherein the efflux pump expression factor is CaNDT80, or a homolog or variant thereof, and modulates the expression of an efflux pump selected from CDR1, CDR2, and MDR1, or a homolog or a variant thereof.

54. The method of claim 52, further comprising contacting the microbial cell with at least one drug.

55. The composition of claim 52, wherein the microbial cell is selected from a bacterial cell, fungal cell, and yeast cell, and mammalian cell.

56. The method of claim 55, wherein the fungal cell is selected from a Candida species, Aspergillus species, and Cryptococcus species, or mixtures thereof.

57. The method of claim 56, wherein the Candida species is selected from C. albicans, C. glabrata, C. krusei, and C. tropicalis, or mixtures thereof.

58. The method of claim 55, wherein the mammalian cell is a cancer cell.
